(19) United States
(12) Patent Application Publication
(10) Pub. No.: US 2024/0018202 A1
(43) Pub. Date: Jan. 18, 2024

(54) COMPOSITIONS AND METHODS FOR IMPROVING LENTIVIRAL TRANSDUCTION

(71) Applicant: Donaldson Company, Inc., Bloomington, MN (US)

(72) Inventors: Nicole VOTAW, Durham, NC (US); Michael DZURICKY, Durham, NC (US); Kelli M. LUGINBUHL, Durham, NC (US)

(21) Appl. No.: 18/351,209

(22) Filed: Jul. 12, 2023

Related U.S. Application Data
(60) Provisional application No. 63/388,544, filed on Jul. 12, 2022.

(51) Int. Cl. C07K 14/47; C12N 15/86

(52) U.S. Cl. CPC C07K 14/4702; C12N 15/86; C12N 2740/15043; C07K 2319/00

(57) ABSTRACT

Disclosed herein are fusion proteins comprising the CR3 domain of the low density lipoprotein (LDLR) and a polypeptide with phase behavior. Also provided herein are polypeptides with phase behavior. Additionally provided herein are methods of improving or decreasing viral transduction efficiency comprising administering a fusion protein or polypeptide with phase behavior described herein.

### CROSS REFERENCE TO RELATED APPLICATIONS

This application claims the benefit of U.S. Provisional Application No. 63/388,544, filed Jul. 12, 2022, which is hereby incorporated by reference in its entirety.

### REFERENCE TO AN ELECTRONIC SEQUENCE LISTING

The contents of the electronic sequence listing (ISOL_010_01US_SeqList_ST26.xml; Size: 54,873 bytes; and Date of Creation: Jul. 11, 2023) are herein incorporated by reference in its entirety.

### FIELD

The present disclosure is generally related to compositions and methods for improving lentiviral transduction efficiency. The disclosure also provides fusion proteins comprising the CR3 domain of the LDLR and polypeptides with phase behavior, and methods of using the same to improve lentiviral transduction efficiency. The disclosure is also related to using polypeptides with phase behavior to improve lentiviral transduction efficiency.

### BACKGROUND OF THE INVENTION

Lentiviral transduction is used to genetically engineer therapeutic cells. For example, lentiviral transduction is used to generate T cells, such as chimeric antigen receptor (CAR) T cells, which express a chimeric antigen receptor on their surface that targets antigens on tumor cells, resulting in tumor cell death. These therapeutics are lifesaving. However, transduction efficiency can be low, which results in higher manufacturing costs. There is a need in the art for compositions and methods for improving lentiviral transduction efficiency to decrease the cost of CAR T therapeutics. The present disclosure solves the need in the art for compositions and methods that improve.

### SUMMARY OF THE INVENTION

Provided herein are compositions comprising fusion proteins comprising the CR3 domain of the low density lipoprotein receptor (LDLR) and polypeptides with phase behavior. The compositions described herein unexpectedly improve or block lentiviral transduction depending on the identity of the polypeptide with phase behavior.

Provided herein is a fusion protein comprising the CR3 domain of the low density lipoprotein (LDLR) that is at least 80% identical to the polypeptide of SEQ ID NO: 14 and a polypeptide with phase behavior that is at least 80% identical to a polypeptide of any one of SEQ ID NOS: 1, 2, or 11-13. In embodiments, the CR3 domain of the LDLR comprises a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 14. In embodiments, the CR3 domain of the LDLR comprises the polypeptide sequence of SEQ ID NO: 14. In embodiments, the polypeptide with phase behavior comprises a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 2, or 11-13. In embodiments, the polypeptide with phase behavior comprises the polypeptide sequence of SEQ ID NO: 1. In embodiments, the polypeptide with phase behavior comprises the polypeptide sequence of SEQ ID NO: 2. In embodiments, the polypeptide with phase behavior comprises the polypeptide sequence of SEQ ID NO: 11. In embodiments, the polypeptide with phase behavior is N-terminal to the CR3 domain of the LDLR (e.g., the polypeptide with phase behavior is attached at the N-terminus of the CR3 domain of the LDLR). In embodiments, the polypeptide with phase behavior is C-terminal to the CR3 domain of the LDLR (e.g., the polypeptide with phase behavior is attached at the C-terminus of the CR3 domain of the LDLR). In embodiments, the fusion protein comprises a linker between the polypeptide with phase behavior and the CR3 domain of the LDLR. In embodiments, the linker has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 21-38.

Provided herein is a method of improving lentiviral transduction efficiency, comprising transducing a cell with a lentivirus and administering a fusion protein described herein to the cell. In embodiments, the lentiviral transduction efficiency is improved compared to the lentiviral transduction efficiency of a method that does not comprise administering a fusion protein described herein to the cell transduced with a lentivirus. In embodiments, the lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%. In embodiments, the method comprises administering a fusion protein comprising a polypeptide with phase behavior with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 11. In embodiments, the method comprises administering a fusion protein having the amino acid sequence of SEQ ID NO: 11. In embodiments, the method comprises administering a fusion protein having an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 5. In embodiments, the method comprises administering a fusion protein having an amino acid sequence of SEQ ID NO: 5. In embodiments, the method improves lentiviral transduction efficiency by at least 120%.

In embodiments, the method of improving lentiviral transduction comprises transducing a cell with a lentivirus and administering a polypeptide with phase behavior to the cell; wherein the amino acid sequence of the polypeptide with phase behavior is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. In embodiments, the method of improving lentiviral transduction comprises transducing a cell with a lentivirus and administering a polypeptide with phase behavior to the cell; wherein the polypeptide with phase behavior has the amino acid sequence of SEQ ID NO: 2. In embodiments, the method improves the lentiviral transduction efficiency by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%. In embodiments, the method improves the lentiviral transduction efficiency by at least 90%. In embodiments, the method comprises improving the lentiviral transduction efficiency compared to the lentiviral transduction efficiency of a method, which does not comprise administering the polypeptide with phase behavior to a cell transduced with lentivirus.

Provided herein are fusion proteins comprising the CR3 domain of the low density lipoprotein (LDLR) that is at least 80% identical to the polypeptide of SEQ ID NO: 14 and a polypeptide with phase behavior that is at least 80% identical to a polypeptide of any one of SEQ ID NOS: 1, 2, 11-13, and 41-42. In embodiments, the CR3 domain of the LDLR of the fusion protein comprises a polypeptide that is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 14. In embodiments, the CR3 domain of the LDLR comprises the polypeptide sequence of SEQ ID NO: 14. In embodiments, the polypeptide with phase behavior of the fusion protein comprises a polypeptide that is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 2, 11-13, and 41-42. In embodiments, the polypeptide with phase behavior of the fusion protein comprises the polypeptide sequence of any one of SEQ ID NO: 1, SEQ ID NO: 12, SEQ ID NO: 13, and SEQ ID NO: 42. In embodiments, the fusion protein comprises a linker between the polypeptide with phase behavior and the CR3 domain of the LDLR. In embodiments, the linker has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 21-38. In embodiments, the fusion protein has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of SEQ ID NO: 5.

Provided herein are methods of improving lentiviral transduction efficiency, comprising transducing a cell with a lentivirus and administering a fusion protein described herein, wherein lentiviral transduction of the cells administered the lentivirus and the fusion protein is improved as compared to cells that are transduced with the lentivirus and not administered the fusion protein. In embodiments, lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%. In embodiments, the lentiviral transduction efficiency is improved from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 20% to about 200%, from about 30% to about 200%, from about 40% to about 200%, from about 50% to about 200%, from about 60% to about 200%, from about 70% to about 200%, from about 80% to about 200%, from about 90% to about 200%, from about 100% to about 200%, from about 110% to about 200%, from about 120% to about 200%, from about 130% to about 200%, from about 140% to about 200%, from about 150% to about 200%, from about 160% to about 200%, from about 170% to about 200%, from about 180% to about 200%, from about 190% to about 200%, from about 20% to about 300%, from about 30% to about 300%, from about 40% to about 300%, from about 50% to about 300%, from about 60% to about 300%, from about 70% to about 300%, from about 80% to about 300%, from about 90% to about 300%, from about 100% to about 300%, from about 110% to about 300%, from about 120% to about 300%, from about 130% to about 300%, from about 140% to about 300%, from about 150% to about 300%, from about 160% to about 300%, from about 170% to about 300%, from about 180% to about 300%, from about 190% to about 300%, from about 200% to about 300%, from about 210% to about 300%, from about 220% to about 300%, from about 230% to about 300%, from about 240% to about 300%, from about 250% to about 300%, from about 260% to about 300%, from about 270% to about 300%, from about 280% to about 300%, from about 290% to about 300%, from about 300% to about 300%, from about 20% to about 400%, from about 30% to about 400%, from about 40% to about 400%, from about 50% to about 400%, from about 60% to about 400%, from about 70% to about 400%, from about 80% to about 400%, from about 90% to about 400%, from about 100% to about 400%, from about 110% to about 400%, from about 120% to about 400%, from about 130% to about 400%, from about 140% to about 400%, from about 150% to about 400%, from about 160% to about 400%, from about 170% to about 400%, from about 180% to about 400%, from about 190% to about 400%, from about 200% to about 400%, from about 210% to about 400%, from about 220% to about 400%, from about 230% to about 400%, from about 240% to about 400%, from about 250% to about 400%, from about 260% to about 400%, from about 270% to about 400%, from about 280% to about 400%, from about 290% to about 400%, from about 300% to about 400%, from about 310% to about 400%, from about 320% to about 400%, from about 330% to about 400%, from about 340% to about 400%, from about 350% to about 400%, from about 360% to about 400%, from about 370% to about 400%, from about 380% to about 400%, from about 390% to about 400%, from about 20% to about 500%, from about 30% to about 500%, from about 40% to about 500%, from about 50% to about 500%, from about 60% to about 500%, from about 70% to about 500%, from about 80% to about 500%, from about 90% to about 500%, from about 100% to about 500%, from about 110% to about 500%, from about 120% to about 500%, from about 130% to about 500%, from about 140% to about 500%, from about 150% to about 500%, from about 160% to about 500%, from about 170% to about 500%, from about 180% to about 500%, from about 190% to about 500%, from about 200% to about 500%, from about 210% to about 500%, from about 220% to about 500%, from about 230% to about 500%, from about 240% to about 500%, from about 250% to about 500%, from about 260% to about 500%, from about 270% to about 500%, from about 280% to about 500%, from about 290% to about 500%, from about 300% to about 500%, from about 310% to about 500%, from about 320% to about 500%, from about 330% to about 500%, from about 340% to about 500%, from about 350% to about 500%, from about 360% to about 500%, from about 370% to about 500%, from about 380% to about 500%, from about 390% to about 500%, from about 400% to about 500%, from about 410% to about 500%, from about 420% to about 500%, from about 430% to about 500%, from about 440% to about 500%, from about 450% to about 500%, from about 460% to about 500%, from about 470% to about 500%, from about 480% to about 500%, or from about 490% to about 500%, wherein lentiviral transduction of the cells administered the lentivirus and the fusion protein is improved as compared to cells that are transduced with the lentivirus and not administered the fusion protein.

In embodiments, the method comprises administering a fusion protein comprising a polypeptide with phase behavior with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 12 or SEQ ID NO: 42. In embodiments, the method comprises administering a fusion protein comprising a polypeptide with phase behavior having the amino acid sequence of SEQ ID NO: 12 or SEQ ID NO: 42.

In embodiments, the method comprises administering a fusion protein comprising a CR3 domain of LDLR with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of SEQ ID NO: 14. In embodiments, the method comprises administering a fusion protein having an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 5. In embodiments, the lentiviral transduction efficiency is improved by at least 120%.

Provided herein are methods of improving lentiviral transduction efficiency, comprising transducing a cell with a lentivirus and administering a polypeptide with phase behavior; wherein the amino acid sequence of the polypeptide with phase behavior is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12, wherein lentiviral transduction of the cells administered the lentivirus and the polypeptide with phase behavior is improved as compared to cells that are administered the lentivirus and not administered the polypeptide with phase behavior. In embodiments, the lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%, wherein lentiviral transduction of the cells administered the lentivirus and the polypeptide with phase behavior is improved as compared to cells that are administered the lentivirus and not administered the polypeptide with phase behavior. In embodiments, the lentiviral transduction efficiency is improved from about 10% to about 100%, from about 20% to about 100%, from about 30% to about 100%, from about 40% to about 100%, from about 50% to about 100%, from about 60% to about 100%, from about 70% to about 100%, from about 80% to about 100%, from about 90% to about 100%, from about 20% to about 200%, from about 30% to about 200%, from about 40% to about 200%, from about 50% to about 200%, from about 60% to about 200%, from about 70% to about 200%, from about 80% to about 200%, from about 90% to about 200%, from about 100% to about 200%, from about 110% to about 200%, from about 120% to about 200%, from about 130% to about 200%, from about 140% to about 200%, from about 150% to about 200%, from about 160% to about 200%, from about 170% to about 200%, from about 180% to about 200%, from about 190% to about 200%, from about 20% to about 300%, from about 30% to about 300%, from about 40% to about 300%, from about 50% to about 300%, from about 60% to about 300%, from about 70% to about 300%, from about 80% to about 300%, from about 90% to about 300%, from about 100% to about 300%, from about 110% to about 300%, from about 120% to about 300%, from about 130% to about 300%, from about 140% to about 300%, from about 150% to about 300%, from about 160% to about 300%, from about 170% to about 300%, from about 180% to about 300%, from about 190% to about 300%, from about 200% to about 300%, from about 210% to about 300%, from about 220% to about 300%, from about 230% to about 300%, from about 240% to about 300%, from about 250% to about 300%, from about 260% to about 300%, from about 270% to about 300%, from about 280% to about 300%, from about 290% to about 300%, from about 300% to about 300%, from about 20% to about 400%, from about 30% to about 400%, from about 40% to about 400%, from about 50% to about 400%, from about 60% to about 400%, from about 70% to about 400%, from about 80% to about 400%, from about 90% to about 400%, from about 100% to about 400%, from about 110% to about 400%, from about 120% to about 400%, from about 130% to about 400%, from about 140% to about 400%, from about 150% to about 400%, from about 160% to about 400%, from about 170% to about 400%, from about 180% to about 400%, from about 190% to about 400%, from about 200% to about 400%, from about 210% to about 400%, from about 220% to about 400%, from about 230% to about 400%, from about 240% to about 400%, from about 250% to about 400%, from about 260% to about 400%, from about 270% to about 400%, from about 280% to about 400%, from about 290% to about 400%, from about 300% to about 400%, from about 310% to about 400%, from about 320% to about 400%, from about 330% to about 400%, from about 340% to about 400%, from about 350% to about 400%, from about 360% to about 400%, from about 370% to about 400%, from about 380% to about 400%, from about 390% to about 400%, from about 20% to about 500%, from about 30% to about 500%, from about 40% to about 500%, from about 50% to about 500%, from about 60% to about 500%, from about 70% to about 500%, from about 80% to about 500%, from about 90% to about 500%, from about 100% to about 500%, from about 110% to about 500%, from about 120% to about 500%, from about 130% to about 500%, from about 140% to about 500%, from about 150% to about 500%, from about 160% to about 500%, from about 170% to about 500%, from about 180% to about 500%, from about 190% to about 500%, from about 200% to about 500%, from about 210% to about 500%, from about 220% to about 500%, from about 230% to about 500%, from about 240% to about 500%, from about 250% to about 500%, from about 260% to about 500%, from about 270% to about 500%, from about 280% to about 500%, from about 290% to about 500%, from about 300% to about 500%, from about 310% to about 500%, from about 320% to about 500%, from about 330% to about 500%, from about 340% to about 500%, from about 350% to about 500%, from about 360% to about 500%, from about 370% to about 500%, from about 380% to about 500%, from about 390% to about 500%, from about 400% to about 500%, from about 410% to about 500%, from about 420% to about 500%, from about 430% to about 500%, from about 440% to about 500%, from about 450% to about 500%, from about 460% to about 500%, from about 470% to about 500%, from about 480% to about 500%, or from about 490% to about 500%, wherein lentiviral transduction of the cells administered the lentivirus and the polypeptide with phase behavior is improved as compared to cells that are administered the lentivirus and not administered the polypeptide with phase behavior.

In embodiments, the lentiviral transduction efficiency is improved by at least 90%. In embodiments, the polypeptide with phase behavior has the amino acid sequence of SEQ ID NO: 12.

These and other embodiments will be further described below in the Detailed Description, Examples, and Claims.

### BRIEF DESCRIPTION OF THE DRAWINGS

FIG. 1 shows GFP expression (as determined by flow cytometry) in HEK293T cells that were transduced with lentivirus expressing GFP and incubated with Reagent 1, 2, 3, 4, or 5. Fusing the CR3 domain of LDLR to different polypeptides with phase behavior enhances or blocks transduction of lentiviral particles. Reagents 1 and 4 had no effect on transduction efficiency, while reagents 2 and 5 significantly enhanced transduction efficiency and reagent 3 significantly blocked lentiviral transduction. Control indicates samples spiked with lentiviral particles without reagent added. ***p<0.001, ****p<0.0001 by one-way ANOVA with Dunnett's post hoc test. Mean+/−s.e.m. shown. n=6-9 experimental replicates per group.

FIG. 2 shows the amount of lentiviral-associated p24 in the supernatant of HEK293T cells that were transduced with lentivirus expressing GFP and incubated with Reagent 1, 2, 3, 4, or 5. All samples have similar or higher levels of lentiviral particles in the supernatant than the control indicating virus is retained in the supernatant without incurring damage. Control indicates samples spiked with lentiviral particles without reagent added. *p<0.05, by one-way ANOVA with Dunnett's post hoc test. Mean+/−s.e.m. shown. n=2-3 experimental replicates per group.

FIGS. 3A-3B show GFP expression (as determined by flow cytometry) in HEK293T cells that were transduced with lentivirus expressing GFP co-incubated with Reagent 4 or 5 after mixture was incubated in 1.5 M NaCl for 0, 1, 2, 4, 6, or 12 hours. Lentivirus was produced in either adherent, serum-containing (FIG. 3A) or suspension, serum free media (FIG. 3B). Control indicates samples spiked with lentiviral particles without reagent added. *p<0.05, **p<0.01, ***p<0.001 by two-way ANOVA with Fisher's LSD post hoc test. Mean+/−s.e.m. shown. n=4 replicates per group.

FIGS. 4A-4B show GFP expression (as determined by flow cytometry) in HEK293T cells that were transduced with lentivirus expressing GFP co-incubated with Reagent 4 or 5 after the mixture was stored at 4-8° C. for 0, 1, 2, 3, 4, or 5 weeks. Lentivirus was produced in either adherent, serum-containing (FIG. 4A) or suspension, serum free media (FIG. 4B). Control indicates samples spiked with lentiviral particles without reagent added. *p<0.05, **p<0.01, ***p<0.001 by two-way ANOVA with Fisher's LSD post hoc test. Mean+/−s.e.m. shown. n=4 replicates per group.

FIGS. 5A-5B show GFP expression (as determined by flow cytometry) in HEK293T cells that were transduced with lentivirus expressing GFP co-incubated with Reagent 4 or 5 after mixture was stored at 37° C. for 0, 2, 4, or 6 hours. Lentivirus was produced in either adherent, serum-containing (FIG. 5A) or suspension, serum free media (FIG. 5B). Control indicates samples spiked with lentiviral particles without reagent added. *p<0.05, **p<0.01, ***p<0.001 by two-way ANOVA with Fisher's LSD post hoc test. Mean+/−s.e.m. shown. n=4 replicates per group.

FIG. 6 shows GFP expression (as determined by flow cytometry) in HEK293T cells that were transduced with lentivirus expressing GFP co-incubated with Reagent 4 or 5 after mixture was stored at −80° C. for 0, 1, 2, 3, 4, or 5 freeze-thaw cycles. Lentivirus was produced in adherent, serum-containing media. Control indicates samples spiked with lentiviral particles without reagent added. *p<0.05, **p<0.01, ***p<0.001 by two-way ANOVA with Fisher's LSD post hoc test. Mean+/−s.e.m. shown. n=4 replicates per group.

FIGS. 7A-7B show GFP expression (as determined by flow cytometry) in HEK293T cells that were transduced with adenovirus expressing GFP and incubated with Reagent 1 or 2. Control indicates samples spiked with adenoviral particles without reagent added. n=3 technical replicates per group.

### DETAILED DESCRIPTION OF THE INVENTION

### Definitions

As used herein, and in the appended claims, the singular forms “a”, “an”, and “the” include plural referents unless the context clearly dictates otherwise. Thus, for example, reference to “a protein” can refer to one protein or to mixtures of such protein, and reference to “the method” includes reference to equivalent steps and/or methods known to those skilled in the art, and so forth.

As used herein, the term “about” or “approximately” when preceding a numerical value indicates the value plus or minus a range of 10%. For example, “about 100” encompasses 90 and 110.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination.

Moreover, the present disclosure also contemplates that in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in particular embodiments the amino acid is not A, G or I; is not A; is not G or V; etc., as if each such possible disclaimer is expressly set forth herein.

As used herein, the term “fragment” as it refers to a protein or polypeptide includes a truncated form of the protein or polypeptide. For example, a fragment of LDLR may include about 20%, about 25%, about 30%, about 35%, about 40%, about 45%, about 50%, about 55%, about 60%, about 65%, about 70%, about 75%, about 80%, about 85%, about 90%, about 95%, about 97%, or about 99% of the amino acids of full-length LDLR. A fragment of the CR3 domain of LDLR may contain about 20%, about 25%, about 30%, about 35%, about 40%, about 45%, about 50%, about 55%, about 60%, about 65%, about 70%, about 75%, about 80%, about 85%, about 90%, about 95%, about 97%, or about 99% of the amino acids of the full-length CR3 domain of LDLR. The full-length CR3 domain of LDLR has the amino acid sequence of SEQ ID NO: 14. In embodiments, the fragment of the CR3 domain of LDLR comprises at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, or at least 38 amino acids of the CR3 domain of LDLR.

As used herein, the terms “peptide,” “polypeptide,” and “protein” are used interchangeably, and refer to a compound comprised of amino acid residues covalently linked by peptide bonds. A protein must contain at least two amino acids, and no limitation is placed on the maximum number of amino acids that can comprise a protein's sequence. The term “peptide” may refer to a short chain of amino acids including, for example, natural peptides, recombinant peptides, synthetic peptides, or a combination thereof. Proteins and peptides may include, for example, biologically active fragments, substantially homologous polypeptides, oligopeptides, homodimers, heterodimers, variants of polypeptides, modified polypeptides, derivatives, analogs, and fusion proteins, among others.

The term “modification” as it refers to a polypeptide refers to mutation, deletion, or addition of one amino acid of the polypeptide. In embodiments, the polypeptide is the CR3 domain of the LDLR. The location of a modification within the CR3 domain of the LDLR can be determined based on aligning the sequence of the polypeptide to SEQ ID NO: 14 (the amino acid sequence of the CR3 domain of the LDLR).

A “polynucleotide” is a sequence of nucleotide bases, and may be RNA, DNA or DNA-RNA hybrid sequences (including both naturally occurring and non-naturally occurring nucleotides). In some embodiments, a polynucleotide is either a single or double stranded DNA sequence.

The term “percent identity” in the context of two or more nucleic acid or polypeptide sequences, refers to two or more sequences or subsequences that have a specified percentage of nucleotides or amino acid residues that are the same, when compared. Unless otherwise indicated, percent identity is determined using the EMBL's European Bioinformatic's Institute (EMBL-EBI) tool EMBOSS Needle, which is available at https://www.ebi.ac.uk/Tools/psa/emboss_needle/. The following default parameters may be used for EMBOSS Needle Pairwise Alignment: Matrix=BLOSUM62; Gap Open=10; Gap Extension=0.5; End Gap Penalty=false; End Gap Open=10; End Gap Extend=0.5. In embodiments, the percent identity is calculated over the entire length of the compared sequences. In some embodiments, the sequence identity is calculated over a fragment of each compared sequence of about 10 amino acids, about 15 amino acids, about 20 amino acids, about 25 amino acids, about 30 amino acids, about 35 amino acids, about 40 amino acids, about 45 amino acids, about 50 amino acids, about 55 amino acids, about 60 amino acids, about 65 amino acids, about 70 amino acids, about 75 amino acids, about 80 amino acids, about 85 amino acids, about 90 amino acids, about 95 amino acids, about 100 amino acids, about 105 amino acids, about 110 amino acids, about 115 amino acids, about 120 amino acids, about 125 amino acids, about 130 amino acids, about 135 amino acids, about 140 amino acids, about 145 amino acids, about 150 amino acids, about 155 amino acids, about 160 amino acids, about 165 amino acids, about 170 amino acids, about 175 amino acids, about 180 amino acids, about 185 amino acids, about 190 amino acids, about 195 amino acids, or about 200 amino acids.

Compositions Comprising Fusion Proteins Comprising the CR3 domain of LDLR and a Polypeptide with Phase Behavior

The disclosure provides compositions comprising fusion proteins comprising the CR3 domain of LDLR and a polypeptide with phase behavior. In embodiments, the CR3 domain of LDLR is a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 14. In embodiments, the CR3 domain of LDLR contains from about 20 to about 39, from about 21 to about 39, from about 22 to about 39, from about 23 to about 39, from about 24 to about 39, from about 25 to about 39, from about 26 to about 39, from about 27 to about 39, from about 28 to about 39, from about 29 to about 39, from about 30 to about 39, from about 31 to about 39, from about 32 to about 39, from about 33 to about 39, from about 34 to about 39, from about 35 to about 39, from about 36 to about 39, from about 37 to about 39, or from about 38 to about 39 contiguous amino acids of SEQ ID NO: 14. In embodiments, the CR3 domain of LDLR contains about 20, about 21, about 22, about 23, about 24, about 25, about 26, about 27, about 28, about 29, about 30, about 31, about 32, about 33, about 34, about 35, about 36, about 37, about 38, or about 39 contiguous amino acids of SEQ ID NO: 14. In embodiments, the CR3 domain of LDLR comprises from 1 to 5 modifications, from 1 to 4, from 1 to 3, or from 1 to 2 modifications compared to a polypeptide of SEQ ID NO: 14. In embodiments, the CR3 domain of LDLR comprises 1, 2, 3, 4, or 5 modifications compared to a polypeptide of SEQ ID NO: 14.

In embodiments, the polypeptide with phase behavior has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to a polypeptide of any one of SEQ ID NOS: 1, 2, 11-13, 39, and 41-42. In embodiments, the polypeptide with phase behavior has 1, 2, 3, 4, 5, 6, 7, 8, 9, or 10 modifications compared to a polypeptide of any one of SEQ ID NOS: 1, 2, 11-13, 39, and 41-42. In embodiments, the polypeptide with phase behavior comprises from about 1 to about 5, from about 1 to about 6, from about 1 to about 7, from about 1 to about 8, from 1 to about 9, or from 1 to about 10 modifications compared to a polypeptide of any one of SEQ ID NOS: 1, 2, 11-13, 39, and 41-42.

In embodiments, the polypeptide with phase behavior comprises the amino acid sequence of SEQ ID NO: 1. In embodiments, the polypeptide with phase behavior comprises the amino acid sequence of SEQ ID NO: 2. In embodiments, the polypeptide with phase behavior comprises the amino acid sequence of SEQ ID NO: 11. In embodiments, the polypeptide with phase behavior comprises the amino acid sequence of SEQ ID NO: 12. In embodiments, the polypeptide with phase behavior comprises the amino acid sequence of SEQ ID NO: 13. In embodiments, the polypeptide with phase behavior comprises the amino acid sequence of SEQ ID NO: 39. In embodiments, the polypeptide with phase behavior comprises the amino acid sequence of SEQ ID NO: 41. In embodiments, the polypeptide with phase behavior comprises the amino acid sequence of SEQ ID NO: 42.

In embodiments, the fusion protein is a polypeptide with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to any one of SEQ ID NOS: 3-5 and 15-20. In embodiments, the fusion protein is a polypeptide with 1, 2, 3, 4, 5, 6, 7, 8, 9, or 10 modifications compared to a polypeptide of any one of SEQ ID NOS: 3-5 and 15-20. In embodiments, the fusion protein comprises from about 1 to about 5, from about 1 to about 6, from about 1 to about 7, from about 1 to about 8, from 1 to about 9, or from 1 to about 10 modifications compared to a polypeptide of any one of SEQ ID NOS: 3-5 and 15-20.

In embodiments, the fusion protein comprises the amino acid sequence of SEQ ID NO: 3. In embodiments, the fusion protein comprises the amino acid sequence of SEQ ID NO: 5.

In embodiments, the fusion protein comprises a polypeptide with phase behavior of SEQ ID NO: 1 and the CR3 domain of LDLR (SEQ ID NO: 14). In embodiments, the fusion protein comprises a polypeptide with phase behavior of SEQ ID NO: 2 and the CR3 domain of LDLR (SEQ ID NO: 14). In embodiments, the fusion protein comprises a polypeptide with phase behavior of SEQ ID NO: 11 and the CR3 domain of LDLR (SEQ ID NO: 14). In embodiments, the fusion protein comprises a polypeptide with phase behavior of SEQ ID NO: 12 and the CR3 domain of LDLR (SEQ ID NO: 14). In embodiments, the fusion protein comprises a polypeptide with phase behavior of SEQ ID NO: 13 and the CR3 domain of LDLR (SEQ ID NO: 14). In embodiments, the fusion protein comprises a polypeptide with phase behavior of SEQ ID NO: 42 and the CR3 domain of LDLR (SEQ ID NO: 14).

In embodiments, provided herein are nucleic acids encoding the fusion proteins described herein. In embodiments, provided herein are nucleic acids encoding the polypeptides with phase behavior described herein. In embodiments, the polypeptide with phase behavior is encoded by a nucleic acid of any one of SEQ ID NOS: 6-8, or a nucleic acid with at least 90%, at least 91%, at least 92%, at least 93%, at least 94%, at least 95%, at least 96%, at least 97%, at least 98%, or at least 99% identity to any one of SEQ ID NOS: 6-8. In embodiments, the fusion protein is encoded by a nucleic acid of any one of SEQ ID NOS: 9-10, or a nucleic acid with at least 90%, at least 91%, at least 92%, at least 93%, at least 94%, at least 95%, at least 96%, at least 97%, at least 98%, or at least 99% identity to any one of SEQ ID NOS: 9-10.

In embodiments, the fusion protein comprises an N-terminal methionine. In embodiments, the fusion protein lacks an N-terminal methionine.

In embodiments, the fusion protein comprises a linker between the polypeptide with phase behavior and the CR3 domain of LDLR. In embodiments, the linker does not interfere with the function of a fusion protein described herein. In some embodiments, the linker may adopt various secondary structures, including but not limited to α-helices, β-strands, and random coils. In some embodiments, the linker adopts an α-helix and comprises an amino acid repeat of (EAAAK)ₙ (SEQ ID NO: 21) where n is an integer from 1 to 20.

In some embodiments, the linker is comprised of (G₄S)ₙ(SEQ ID NO: 22) where n can be an integer from 1 to 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30). In embodiments, the polypeptide linker has a repeat of (SGGG)n (SEQ ID NO: 23), wherein n is an integer from 1 to 50 (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50). In embodiments, the polypeptide linker has a repeat of (GGGS)ₙ (SEQ ID NO: 24), wherein n is an integer from 1 to 20 (e.g. 1, 2, 3, 4, 5, 6, 7, 8, 9, 10, 11, 12, 13, 14, 15, 16, 17, 18, 19, or 20).

In some embodiments, the linker has an amino acid sequence of KESGSVSSEQLAQFRSLD (SEQ ID NO: 25). In some embodiments, the linker has an amino acid sequence of EGKSSGSGSESKST (SEQ ID NO: 26). In some embodiments, the linker only contains glycine.

In some embodiments, the peptide linker comprises a protease cleavage site. In some embodiments, the protease cleavage site is a furin cleavage site.

In some aspects, the polypeptide linker is a poly-(Gly)ₙ linker, wherein n is an integer from 1 to 30 (e.g., 1, 2, 3, 4, 5, 6, 7, 8, 9, 10, 15, 16, 17, 18, 19, 20, 21, 22, 23, 24, 25, 26, 27, 28, 29, or 30 (SEQ ID NO: 27). In other embodiments, the linker is selected from the group consisting of: dipeptides, tripeptides, and tetrapeptides. In embodiments, the linker is a dipeptide selected from the group consisting of alanine-serine (AS), leucine-glutamic acid (LE), and serine-arginine (SR).

In some embodiments, the linker is selected from GKSSGSGSESKS (SEQ ID NO: 28), GSTSGSGKSSEGKG (SEQ ID NO: 29), GSTSGSGKSSEGSGSTKG (SEQ ID NO: 30), GSTSGSGKPGSGEGSTKG (SEQ ID NO: 31), EGKSSGSGSESKEF (SEQ ID NO: 32), SRSSG (SEQ ID NO: 33), and SGSSC (SEQ ID NO: 34).

In some embodiments, the linker is a self-cleaving peptide. In some embodiments, the self-cleaving peptide is a 2A peptide. 2A peptides are a class of 18-22 amino acid long peptides that induce ribosomal skipping during translation of a protein in a cell. In some embodiments, the 2A peptide is a T2A peptide having an amino acid sequence of EGRGSLLTCGDVEENPGP (SEQ ID NO: 35), a P2A peptide having an amino acid sequence of ATNFSLLKQAGDVEENPGP (SEQ ID NO: 36), an E2A peptide having an amino acid sequence of QCTNYALLKLAGDVESNPGP (SEQ ID NO: 37), or an F2A peptide having an amino acid sequence of VKQTLNFDLLKLAGDVESNPGP (SEQ ID NO: 38). In some embodiments, the 2A peptide has at least 80%, at least 85%, at least 90%, at least 95%, or at least 98% identity to any one of SEQ ID NOs. 35-38.

In embodiments, the polypeptide with phase behavior is located N-terminal to the CR3 domain of LDLR. (e.g., the polypeptide with phase behavior is attached at the N-terminus of the CR3 domain of LDLR). In embodiments, the polypeptide with phase behavior is located C-terminal to the CR3 domain of LDLR (e.g., the polypeptide with phase behavior is attached at the C-terminus of the CR3 domain of LDLR).

Methods of Utilizing Compositions Comprising Polypeptides with Phase Behavior or Fusion Proteins Comprising the CR3 Domain of LDLR and a Polypeptide with Phase Behavior

In embodiments, provided herein are methods of improving lentiviral transduction efficiency. In embodiments, the method of improving lentiviral transduction efficiency comprises administering a fusion protein described herein to a cell. In embodiments, the fusion protein is administered to a cell simultaneously (i.e., within 15 minutes) with a lentivirus. In embodiments, the fusion protein is administered within 15 minutes, within 30 minutes, within 45 minutes, within an hour, within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of lentivirus. In embodiments, the fusion protein is administered to the cell before lentivirus is administered to the cell. In embodiments, the fusion protein is administered to the cell after lentivirus is administered to the cell. In embodiments, the fusion protein has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS: 3-5 or 15-20.

In embodiments, the method of improving lentiviral transduction efficiency comprises administering a polypeptide with phase behavior to a cell. In embodiments, the polypeptide with phase behavior is administered simultaneously (i.e., within 15 minutes) with a lentivirus. In embodiments, the polypeptide with phase behavior is administered within 15 minutes, within 30 minutes, within 45 minutes, within an hour, within 2 hours, within 3 hours, within 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of lentivirus. In embodiments, the polypeptide with phase behavior is administered to the cell before lentivirus is administered to the cell. In embodiments, the polypeptide with phase behavior is administered to the cell after lentivirus is administered to the cell. In embodiments, the polypeptide with phase behavior has a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOS: 1, 2, or 11-13.

In embodiments, lentiviral transduction efficiency (or the percentage of cells that are infected with lentivirus) is evaluated by flow cytometry. For example, in embodiments, a lentivirus encodes a fluorescent protein, such as green fluorescent protein. The lentiviral transduction efficiency is the percentage of cells that express the fluorescent protein. In other embodiments, the lentivirus encodes a cell surface protein. Cells transduced with lentivirus encoding the cell surface protein may be incubated with a fluorescent antibody that binds to the cell surface protein. The lentiviral transduction efficiency is the percentage of cells with bound fluorescent antibody.

In embodiments, the methods described herein provide for improved lentiviral transduction efficiency compared to an alternative method. In embodiments, the alternative method comprises a lentiviral transduction method, which does not administer a fusion protein or polypeptide with phase behavior described herein. In embodiments, the alternative method is a conventional method for improving lentiviral transduction. Examples of conventional methods for improving lentiviral transduction include transducing in the presence of polybrene or the lentivirus enhancer TRANSDUX™ MAX.

In embodiments, the methods described herein improve lentiviral transduction efficiency by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500% compared to an alternative method.

In embodiments, the method for improving lentiviral transduction efficiency comprises administering a polypeptide with phase behavior with an amino acid sequence that is at least 90%, at least 91%, at least 92%, at least 93%, at least 94%, at least 95%, at least 96%, at least 97%, at least 98%, at least 99%, or 100% identical to SEQ ID NO: 2. In embodiments, administering a polypeptide with phase behavior with an amino acid sequence that is at least 90%, at least 91%, at least 92%, at least 93%, at least 94%, at least 95%, at least 96%, at least 97%, at least 98%, at least 99%, or 100% identical to SEQ ID NO: 2 improves lentiviral transduction efficiency by at least 90% compared to an alternative method.

In embodiments, the method for improving lentiviral transduction efficiency compromises administering a fusion protein having an amino acid sequence that is at least 90%, at least 91%, at least 92%, at least 93%, at least 94%, at least 95%, at least 96%, at least 97%, at least 98%, at least 99%, or 100% identical to SEQ ID NO: 5. In embodiments, administering a fusion protein having an amino acid sequence that is at least 90%, at least 91%, at least 92%, at least 93%, at least 94%, at least 95%, at least 96%, at least 97%, at least 98%, at least 99%, or 100% identical to SEQ ID NO: 5 improves lentiviral transduction efficiency by at least 120%.

In embodiments, the methods described herein provide for decreased lentiviral transduction efficiency. In embodiments, the lentiviral transduction efficiency of a method for lentivirus transduction described herein, which comprises administering a polypeptide with phase behavior or fusion protein described herein to a cell, is decreased by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500% compared to a method that does not administer a polypeptide with phase behavior or fusion protein described herein to a cell. In embodiments, a method of lentiviral transduction comprising administering a fusion protein having an amino acid sequence that is at least 90%, at least 91%, at least 92%, at least 93%, at least 94%, at least 95%, at least 96%, at least 97%, at least 98%, at least 99%, or 100% identical to SEQ ID NO: 3 decreases lentiviral transduction efficiency compared to a method that does not comprise administering a polypeptide with phase behavior by at least 80%.

In embodiments, the methods described herein provide for decreased adenoviral transduction efficiency. In embodiments, the adenoviral transduction efficiency of a method for adenovirus transduction described herein, which comprises administering a polypeptide with phase behavior described herein, is decreased by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500% compared to a method that does not administer a polypeptide with phase behavior. In embodiments, the method comprises incubating a cell with an adenovirus and a polypeptide with phase behavior. In embodiments, the polypeptide with phase behavior comprises an amino acid sequence that is at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 13, 39, and 41.

### Methods of Stabilizing Lentivirus

In embodiments, provided herein are methods of stabilizing a lentiviral particle. As used herein with relation to a lentiviral particle, the terms “stabilize” or “stabilizing” refers to the ability of a fusion protein to reduce degradation or aggregation of a sample comprising a plurality of lentiviral particles, to prevent lentiviral molecules from binding other proteins, to enhance synthesis of a lentiviral particle by a producer cell, or to otherwise enhance the function of the lentiviral particle. In embodiments, the lentiviral particle that is incubated with a fusion protein described herein is stabilized as compared to a lentiviral particle that is not incubated with a fusion protein described herein in the presence of a condition, such as salt, a freeze-thaw cycle, at about 4-8° C., or at about 37° C.

In embodiments, the method of stabilizing lentivirus comprises incubating the lentivirus with a fusion protein described herein. In embodiments, the fusion protein comprises an amino acid sequence that is at least 90%, at least 91%, at least 92%, at least 93%, at least 94%, at least 95%, at least 96%, at least 97%, at least 98%, at least 99%, or 100% identical to any one of SEQ ID NOS: 4 and 5. In embodiments, fusion protein comprises the CR3 domain of the LDLR. In embodiments, the CR3 domain of the LDLR protein comprises an amino acid sequence that is at least 90%, at least 91%, at least 92%, at least 93%, at least 94%, at least 95%, at least 96%, at least 97%, at least 98%, at least 99%, or 100% identical to SEQ ID NO: 14. In embodiments, the fusion protein comprises a polypeptide with phase behavior comprising an amino acid sequence that is at least 90%, at least 91%, at least 92%, at least 93%, at least 94%, at least 95%, at least 96%, at least 97%, at least 98%, at least 99%, or 100% identical to any one of SEQ ID NOS: 42 and 2.

In embodiments, when the lentivirus is incubated with a fusion protein described herein, the lentivirus is stable in the presence of up to about 1.5 M NaCl for up to about 1, up to about 2, up to about 3, up to about 4, up to about 5, or up to about 6 hours at room temperature. In embodiments, when the lentivirus is incubated with a fusion protein described herein, the lentivirus is stable in the presence of up to about 1.5 M NaCl for at least about 6 hours at room temperature. In embodiments, when the lentivirus is incubated with a fusion protein described herein, the lentivirus is stable in the presence of up to about 1.5 M NaCl for 1 to about 6 hours, for 1 to about 5 hours, for 1 to about 4 hours, for 1 to about 3 hours, or for 1 to about 2 hours. In embodiments, when the lentivirus is incubated with a fusion protein described herein, the lentivirus is stable in the presence of up about 1.5 M NaCl for up to about 1, up to about 2, up to about 3, up to about 4, up to about 5, or up to about 6 hours at room temperature. In embodiments, when the lentivirus is incubated with a fusion protein described herein, the lentivirus is stable in the presence of about 1.5 M NaCl for at least about 6 hours at room temperature. In embodiments, when the lentivirus is incubated with a fusion protein described herein, the lentivirus is stable in the presence of about 1.5 M NaCl for 1 to about 6 hours, for 1 to about 5 hours, for 1 to about 4 hours, for 1 to about 3 hours, or for 1 to about 2 hours.

In embodiments, when the lentivirus is incubated with a fusion protein described herein, the lentivirus is stable at about 4-8° C. for up to about 1, up to about 2, up to about 3, up to about 4, up to about 5, or up to about 6 weeks. In embodiments, when the lentivirus is incubated with a fusion protein described herein, the lentivirus is stable at about 4-8° C. for at least about 1, at least about 2, at least about 3, at least about 4, at least about 5, or at least about 6 weeks. In embodiments, when the lentivirus is incubated with a fusion protein described herein, the lentivirus is stable at about 4-8° C. for 1 to about 6 weeks, for 1 to about 5 weeks, for 1 to about 4 weeks, for 1 to about 3 weeks, or for 1 to about 2 weeks.

In embodiments, when the lentivirus is incubated with a fusion protein described herein, the lentivirus is stable at about 37° C. for up to about 1, up to about 2, up to about 3, up to about 4, up to about 5, or up to about 6 hours. In embodiments, when the lentivirus is incubated with a fusion protein described herein, the lentivirus is stable at about 37° C. for at least about 1, at least about 2, at least about 3, at least about 4, at least about 5, or at least about 6 hours. In embodiments, when the lentivirus is incubated with a fusion protein described herein, the lentivirus is stable at about 37° C. for 1 to about 6 hours, for 1 to about 5 hours, for 1 to about 4 hours, for 1 to about 3 hours, or for 1 to about 2 hours.

In embodiments, when the lentivirus is incubated with a fusion protein described herein, the lentivirus is stable through multiple freeze-thaw cycles. In embodiments, when the lentivirus is incubated with a fusion protein described herein, the lentivirus is stable for at least 1, at least about 2, at least about 3, at least about 4, at least about 5, at least about 6, at least about 7, at least about 8, at least about 9, or at least about 10 freeze thaw cycles. In embodiments, when the lentivirus is incubated with a fusion protein described herein, the lentivirus is stable for 1 to about 10, for 1 to about 9, for 1 to about 8, for 1 to about 7, for 1 to about 6, for 1 to about 5, for 1 to about 4, for 1 to about 3, or for 1 to about 2 freeze-thaw cycles.

### Sequences

In embodiments, provided here is a polypeptide or nucleic acid of Table A. In embodiments, provided here is a polypeptide or nucleic acid with at least 90%, at least 91%, at least 92%, at least 93%, at least 94%, at least 95%, at least 96%, at least 97%, at least 98%, at least 99%, or 100% identity to a polypeptide or nucleic acid of Table A.

TABLE A

| Description | SEQ ID NO: | Sequence (Amino acid N→C; nucleic acid 5′→3′) |
|---|---|---|
| Polypeptide | 1 | GVGVPGLGVPGVGVPGLGVPGVGVPGVGVPGLGVPGVGVPGLGVPGVGVPGVGVPGLG |
| with Phase | | VPGVGVPGLGVPGVGVPGVGVPGLGVPGVGVPGLGVPGVGVPGVGVPGLGVPGVGVPG |
| Behavior | | LGVPGVGVPGVGVPGLGVPGVGVPGLGVPGVGVPGVGVPGLGVPGVGVPGLGVPGVGV |
| (Reagent 1 of | | PGVGVPGLGVPGVGVPGLGVPGVGVPGVGVPGLGVPGVGVPGLGVPGVGVPGVGVPGL |
| Example 1- | | GVPGVGVPGLGVPGVGVPGVGVPGLGVPGVGVPGLGVPGVGVPGVGVPGLGVPGVGVP |
| Amino Acid | | GLGVPGVGVPGVGVPGLGVPGVGVPGLGVPGVGVPGVGVPGLGVPGVGVPGLGVPGVG |
| Sequence; | | VPGVGVPGLGVPGVGVPGLGVPGVGVPGVGVPGLGVPGVGVPGLGVPGVGVPY |
| 40L80) | | |
| Polypeptide | 2 | GVGVPGVGVPGAGVPGVGVPGVGVPGVGVPGVGVPGAGVPGVGVPGVGVPGVGVPGV |
| with Phase | | GVPGAGVPGVGVPGVGVPGVGVPGVGVPGAGVPGVGVPGVGVPGVGVPGVGVPGAGV |
| Behavior | | PGVGVPGVGVPGVGVPGVGVPGAGVPGVGVPGVGVPGVGVPGVGVPGAGVPGVGVPG |
| (Reagent 2 of | | VGVPGVGVPGVGVPGAGVPGVGVPGVGVPGVGVPGVGVPGAGVPGVGVPGVGVPGVG |
| Example 1 - | | VPGVGVPGAGVPGVGVPGVGVPGVGVPGVGVPGAGVPGVGVPGVGVPGVGVPGVGVP |
| Amino Acid | | GAGVPGVGVPGVGVPGVGVPGVGVPGAGVPGVGVPGVGVPGVGVPGVGVPGAGVPGV |
| Sequence; | | GVPGVGVPGVGVPGVGVPGAGVPGVGVPGVGVPGVGVPGVGVPGAGVPGVGVPGVGV |
| 100V80) | | PY |
| Fusion | 3 | GVGVPGLGVPGVGVPGLGVPGVGVPGVGVPGLGVPGVGVPGLGVPGVGVPGVGVPGLG |
| Protein | | VPGVGVPGLGVPGVGVPGVGVPGLGVPGVGVPGLGVPGVGVPGVGVPGLGVPGVGVPG |
| (Reagent 3 of | | LGVPGVGVPGVGVPGLGVPGVGVPGLGVPGVGVPGVGVPGLGVPGVGVPGLGVPGVGV |
| Example 1 | | PGVGVPGLGVPGVGVPGLGVPGVGVPGVGVPGLGVPGVGVPGLGVPGVGVPGVGVPGL |
| Amino Acid | | GVPGVGVPGLGVPGVGVPGVGVPGLGVPGVGVPGLGVPGVGVPGVGVPGLGVPGVGVP |
| Sequence) | | GLGVPGVGVPGVGVPGLGVPGVGVPGLGVPGVGVPGVGVPGLGVPGVGVPGLGVPGVG |
| | | VPGVGVPGLGVPGVGVPGLGVPGVGVPGVGVPGLGVPGVGVPGLGVPGVGVPTCSQDE |
| | | FRCHDGKCISRQFVCDSDRDCLDGSDEASCPGY |
| Fusion | 4 | GVGVPGVGVPGAGVPGVGVPGVGVPGVGVPGVGVPGAGVPGVGVPGVGVPGVGVPGV |
| Protein | | GVPGAGVPGVGVPGVGVPGVGVPGVGVPGAGVPGVGVPGVGVPGVGVPGVGVPGAGV |
| (Reagent 4 of | | PGVGVPGVGVPGVGVPGVGVPGAGVPGVGVPGVGVPGVGVPGVGVPGAGVPGVGVPG |
| Example 1 | | VGVPGVGVPGVGVPGAGVPGVGVPGVGVPGVGVPGVGVPGAGVPGVGVPGVGVPGVG |
| Amino Acid | | VPGVGVPGAGVPGVGVPGVGVPGVGVPGVGVPGAGVPGVGVPGVGVPGVGVPGVGVP |
| Sequence) | | GAGVPGVGVPGVGVPGVGVPGVGVPGAGVPGVGVPGVGVPGVGVPGVGVPGAGVPGV |
| | | GVPGVGVPGVGVPGVGVPGAGVPGVGVPGVGVPGVGVPGVGVPGAGVPGVGVPGVGV |
| | | PTCSQDEFRCHDGKCISRQFVCDSDRDCLDGSDEASCPGY |
| Fusion | 5 | GVGPVGAGPVGVGPVGAGPVGVGPVGVGPVGAGPVGVGPVGAGPVGVGPVGVGPVGA |
| Protein | | GPVGVGPVGAGPVGVGPVGVGPVGAGPVGVGPVGAGPVGVGPVGVGPVGAGPVGVGP |
| (Reagent 5 of | | VGAGPVGVGPVGVGPVGAGPVGVGPVGAGPVGVGPVGVGPVGAGPVGVGPVGAGPVG |
| Example 1 | | VGPVGVGPVGAGPVGVGPVGAGPVGVGPVGVGPVGAGPVGVGPVGAGPVGVGPVGVG |
| Amino Acid | | PVGAGPVGVGPVGAGPVGVGPVGVGPVGAGPVGVGPVGAGPVGVGPVGVGPVGAGPV |
| Sequence) | | GVGPVGAGPVGVGPVGVGPVGAGPVGVGPVGAGPVGVGPVGVGPVGAGPVGVGPVGA |
| | | GPVGVGPVTCSQDEFRCHDGKCISRQFVCDSDRDCLDGSDEASCPGY |
| Reagent 1 of | 6 | TAGCCAGGGACGCCAACACCCGGTACACCCAGACCTGGCACACCGACGCCCGGAACA |
| Example 1 | | CCCAGCCCCGGAACGCCCACACCAGGGACGCCAACACCCGGTACACCCAGACCTGGC |
| (Nucleotide | | ACACCGACGCCCGGAACACCCAGCCCCGGAACGCCCACACCAGGGACGCCAACACCC |
| Sequence) | | GGTACACCCAGACCTGGCACACCGACGCCCGGAACACCCAGCCCCGGAACGCCCACA |
| | | CCAGGGACGCCAACACCCGGTACACCCAGACCTGGCACACCGACGCCCGGAACACCC |
| | | AGCCCCGGAACGCCCACACCAGGGACGCCAACACCCGGTACACCCAGACCTGGCACA |
| | | CCGACGCCCGGAACACCCAGCCCCGGAACGCCCACACCAGGGACGCCAACACCCGGT |
| | | ACACCCAGACCTGGCACACCGACGCCCGGAACACCCAGCCCCGGAACGCCCACACCA |
| | | GGGACGCCAACACCCGGTACACCCAGACCTGGCACACCGACGCCCGGAACACCCAGC |
| | | CCCGGAACGCCCACACCAGGGACGCCAACACCCGGTACACCCAGACCTGGCACACCG |
| | | ACGCCCGGAACACCCAGCCCCGGAACGCCCACACCAGGGACGCCAACACCCGGTACA |
| | | CCCAGACCTGGCACACCGACGCCCGGAACACCCAGCCCCGGAACGCCCACACCAGGG |
| | | ACGCCAACACCCGGTACACCCAGACCTGGCACACCGACGCCCGGAACACCCAGCCCC |
| | | GGAACGCCCACACCAGGGACGCCAACACCCGGTACACCCAGACCTGGCACANCGAC |
| | | GCCCGGAACACCCAGCCCCGGAACGCCCACACCA |
| Reagent 2 of | 7 | TAGCCCGGCACGCCGACACCAGGAACACCAACGCCCGGTACGCCCACACCTGGGACA |
| Example 1 | | CCTACGCCCGGAACACCCACGCCCGGCACGCCGACACCAGGAACACCAACGCCCGGT |
| (Nucleotide | | ACGCCCACACCTGGGACACCTACGCCCGGAACACCCACGCCCGGCACGCCGACACCA |
| Sequence) | | GGAACACCAACGCCCGGTACGCCCACACCTGGGACACCTACGCCCGGAACACCCACG |
| | | CCCGGCACGCCGACACCAGGAACACCAACGCCCGGTACGCCCACACCTGGGACACCT |
| | | ACGCCCGGAACACCCACGCCCGGCACGCCGACACCAGGAACACCAACGCCCGGTACG |
| | | CCCACACCTGGGACACCTACGCCCGGAACACCCACGCCCGGCACGCCGACACCAGGA |
| | | ACACCAACGCCCGGTACGCCCACACCTGGGACACCTACGCCCGGAACACCCACGCCC |
| | | GGCACGCCGACACCAGGAACACCAACGCCCGGTACGCCCACACCTGGGACACCTACG |
| | | CCCGGAACACCCACGCCCGGCACGCCGACACCAGGAACACCAACGCCCGGTACGCCC |
| | | ACACCTGGGACACCTACGCCCGGAACACCCACGCCCGGCACGCCGACACCAGGAACA |
| | | CCAACGCCCGGTACGCCCACACCTGGGACACCTACGCCCGGAACACCCACGCCCGGC |
| | | ACGCCGACACCAGGAACACCAACGCCCGGTACGCCCACACCTGGGACACCTACGCCC |
| | | GGAACACCCACGCCCGGCACGCCGACACCANGAACACCAACGCCCGGTACGCCCACA |
| | | CCTGGG |
| Reagent 3 of | 8 | TAGCCCGGGCAGCTGGCTTCATCGCTGCCATCCAGACAATCGCGATCGCTATCGCAAA |
| Example 1 | | CGAACTGGCGGCTGATGCATTTGCCATCATGACAGCGGAACTCATCTTGGCTGCACGT |
| (Nucleotide | | GCCAGGGACGCCAACACCCGGTACACCCAGACCTGGCACACCGACGCCCGGAACACC |
| Sequence) | | CAGCCCCGGAACGCCCACACCAGGGACGCCAACACCCGGTACACCCAGACCTGGCAC |
| | | ACCGACGCCCGGAACACCCAGCCCCGGAACGCCCACACCAGGGACGCCAACACCCG |
| | | GTACACCCAGACCTGGCACACCGACGCCCGGAACACCCAGCCCCGGAACGCCCACAC |
| | | CAGGGACGCCAACACCCGGTACACCCAGACCTGGCACACCGACGCCCGGAACACCCA |
| | | GCCCCGGAACGCCCACACCAGGGACGCCAACACCCGGTACACCCAGACCTGGCACAC |
| | | CGACGCCCGGAACACCCAGCCCCGGAACGCCCACACCAGGGACGCCAACACCCGGTA |
| | | CACCCAGACCTGGCACACCGACGCCCGGAACACCCAGCCCCGGAACGCCCACACCAN |
| | | GGACGCCAACACCCGGTACACCCAGACCTGGCACACCGACGCCCGGAACACCCAGCC |
| | | CCGGAACGCCCACACCNNGNACGCCAACACCCGGTACACCCAGACCTGGCACACCGA |
| | | CGCCCGGAACACCCAGCCCCGGAACGCCCACACCN |
| Reagent 4 of | 9 | TAGCCCGGGCAGCTGGCTTCATCGCTGCCATCCAGACAATCGCGATCGCTATCGCAAA |
| Example 1 | | CGAACTGGCGGCTGATGCATTTGCCATCATGACAGCGGAACTCATCTTGGCTGCACGT |
| (Nucleotide | | GCCCGGCACGCCGACACCAGGAACACCAACGCCCGGTACGCCCACACCTGGGACACC |
| Sequence) | | TACGCCCGGAACACCCACGCCCGGCACGCCGACACCAGGAACACCAACGCCCGGTAC |
| | | GCCCACACCTGGGACACCTACGCCCGGAACACCCACGCCCGGCACGCCGACACCAGG |
| | | AACACCAACGCCCGGTACGCCCACACCTGGGACACCTACGCCCGGAACACCCACGCC |
| | | CGGCACGCCGACACCAGGAACACCAACGCCCGGTACGCCCACACCTGGGACACCTAC |
| | | GCCCGGAACACCCACGCCCGGCACGCCGACACCAGGAACACCAACGCCCGGTACGCC |
| | | CACACCTGGGACACCTACGCCCGGAACACCCACGCCCGGCACGCCGACACCAGGAAC |
| | | ACCAACGCCCGGTACGCCCACACCTGGGACACCTACGCCCGGAACACCCACGCCCGG |
| | | CACGCCGACACCAGGAACACCAACGCCCGGTACGCCCACACCTGGGACACCTACGCC |
| | | CGGAACACCCACGCCCGGCACGCCGACACCANGAACACCAACGCCCGGTACGCCCAC |
| | | ACCTGGGACACCTACGCCCGGAACACCCACGCCCGGCACGCCGACACCANGAACACC |
| | | AACGCCCGGTACGCCCACACCTGGGACACCTACGCCCGGAACACCCACGCCCGGCAC |
| | | GCCGACACC |
| Reagent 5 of | 10 | TAGCCCGGGCAGCTGGCTTCATCGCTGCCATCCAGACAATCGCGATCGCTATCGCAAA |
| Example 1 | | CGAACTGGCGGCTGATGCATTTGCCATCATGACAGCGGAACTCATCTTGGCTGCACGT |
| (Nucleotide | | GCCAGGAACACCCACACCTGGAACGCCTGCTCCTGGAACACCCACGCCTGGCACTCC |
| Sequence) | | GGCGCCAGGAACACCCACACCTGGAACGCCTGCTCCTGGAACACCCACGCCTGGCAC |
| | | TCCGGCGCCAGGAACACCCACACCTGGAACGCCTGCTCCTGGAACACCCACGCCTGG |
| | | CACTCCGGCGCCAGGAACACCCACACCTGGAACGCCTGCTCCTGGAACACCCACGCC |
| | | TGGCACTCCGGCGCCAGGAACACCCACACCTGGAACGCCTGCTCCTGGAACACCCAC |
| | | GCCTGGCACTCCGGCGCCAGGAACACCCACACCTGGAACGCCTGCTCCTGGAACACC |
| | | CACGCCTGGCACTCCGGCGCCAGGAACACCCACACCTGGAACGCCTGCTCCTGGAAC |
| | | ACCCACGCCTGGCACTCCGGCGCCAGGAACACCCACACCTGGAACGCCTGCTCCTGG |
| | | AACACCCACGCCTGGCACTCCGGCGCCAGGAACACCCACACCTGGAACGCCTGCTCC |
| | | TGGAACACCCACGCCTGGCACTCCGGCGCCAGGAACACCCACACCTGGAACGCCTGC |
| | | TCCTGGAACACCCACGCCTGGCACTCCGGCGCCAGGAACACCCACACCTGGNACGCC |
| | | TGCTCCTGGAACACCCACGCCT |
| Polypeptide | 11 | GVGPVGAGPVGVGPVGAGPVGVGPVGVGPVGAGPVGVGPVGAGPVGVGPV |
| with Phase | | GVGPVGAGPVGVGPVGAGPVGVGPVGVGPVGAGPVGVGPVGAGPVGVGPV |
| Behavior | | GVGPVGAGPVGVGPVGAGPVGVGPVGVGPVGAGPVGVGPVGAGPVGVGPV |
| | | GVGPVGAGPVGVGPVGAGPVGVGPVGVGPVGAGPVGVGPVGAGPVGVGPV |
| | | GVGPVGAGPVGVGPVGAGPVGVGPVGVGPVGAGPVGVGPVGAGPVGVGPV |
| | | GVGPVGAGPVGVGPVGAGPVGVGPVGVGPVGAGPVGVGPVGAGPVGVGPV |
| | | GVGPVGAGPVGVGPVGAGPVGVGPVGVGPVGAGPVGVGPVGAGPVGVGPV |
| | | GVGPVGAGPVGVGPVGAGPVGVGPVGVGPVGAGPVGVGPVGAGPVGVGPV |
| Polypeptide | 12 | GVGVPGVGVPGAGVPGVGVPGVGVPGVGVPGVGVPGAGVPGVGVPGVGVPGVGVPGV |
| with Phase | | GVPGAGVPGVGVPGVGVPGVGVPGVGVPGAGVPGVGVPGVGVPGVGVPGVGVPGAGV |
| Behavior | | PGVGVPGVGVPGVGVPGVGVPGAGVPGVGVPGVGVPGVGVPGVGVPGAGVPGVGVPG |
| | | VGVPGVGVPGVGVPGAGVPGVGVPGVGVPGVGVPGVGVPGAGVPGVGVPGVGVPGVG |
| | | VPGVGVPGAGVPGVGVPGVGVPGVGVPGVGVPGAGVPGVGVPGVGVPGVGVPGVGVP |
| | | GAGVPGVGVPGVGVPGVGVPGVGVPGAGVPGVGVPGVGVPGVGVPGVGVPGAGVPGV |
| | | GVPGVGVPGVGVPGVGVPGAGVPGVGVPGVGVPGVGVPGVGVPGAGVPGVGVPGVGV |
| | | P |
| Polypeptide | 13 | GVGVPGLGVPGVGVPGLGVPGVGVPGVGVPGLGVPGVGVPGLGVPGVGVPGVGVPGLG |
| with Phase | | VPGVGVPGLGVPGVGVPGVGVPGLGVPGVGVPGLGVPGVGVPGVGVPGLGVPGVGVPG |
| Behavior | | LGVPGVGVPGVGVPGLGVPGVGVPGLGVPGVGVPGVGVPGLGVPGVGVPGLGVPGVGV |
| | | PGVGVPGLGVPGVGVPGLGVPGVGVPGVGVPGLGVPGVGVPGLGVPGVGVPGVGVPGL |
| | | GVPGVGVPGLGVPGVGVPGVGVPGLGVPGVGVPGLGVPGVGVPGVGVPGLGVPGVGVP |
| | | GLGVPGVGVPGVGVPGLGVPGVGVPGLGVPGVGVPGVGVPGLGVPGVGVPGLGVPGVG |
| | | VPGVGVPGLGVPGVGVPGLGVPGVGVPGVGVPGLGVPGVGVPGLGVPGVGVP |
| CR3 domain | 14 | TCSQDEFRCHDGKCISRQFVCDSDRDCLDGSDEASCPGY |
| of LDLR | | |
| Fusion | 15 | SKGPGVGVPGLGVPGVGVPGLGVPGVGVPGVGVPGLGVPGVGVPGLGVPGVGVPGVGV |
| Protein | | PGLGVPGVGVPGLGVPGVGVPGVGVPGLGVPGVGVPGLGVPGVGVPGVGVPGLGVPGV |
| | | GVPGLGVPGVGVPGVGVPGLGVPGVGVPGLGVPGVGVPGVGVPGLGVPGVGVPGLGVP |
| | | GVGVPGVGVPGLGVPGVGVPGLGVPGVGVPGVGVPGLGVPGVGVPGLGVPGVGVPGV |
| | | GVPGLGVPGVGVPGLGVPGVGVPGVGVPGLGVPGVGVPGLGVPGVGVPGVGVPGLGVP |
| | | GVGVPGLGVPGVGVPGVGVPGLGVPGVGVPGLGVPGVGVPGVGVPGLGVPGVGVPGLG |
| | | VPGVGVPGVGVPGLGVPGVGVPGLGVPGVGVPGVGVPGLGVPGVGVPGLGVPGVGVPT |
| | | CSQDEFRCHDGKCISRQFVCDSDRDCLDGSDEASCPGY |
| Fusion | 16 | SKGPGVGVPGVGVPGAGVPGVGVPGVGVPGVGVPGVGVPGAGVPGVGVPGVGVPGVG |
| Protein | | VPGVGVPGAGVPGVGVPGVGVPGVGVPGVGVPGAGVPGVGVPGVGVPGVGVPGVGVP |
| | | GAGVPGVGVPGVGVPGVGVPGVGVPGAGVPGVGVPGVGVPGVGVPGVGVPGAGVPGV |
| | | GVPGVGVPGVGVPGVGVPGAGVPGVGVPGVGVPGVGVPGVGVPGAGVPGVGVPGVGV |
| | | PGVGVPGVGVPGAGVPGVGVPGVGVPGVGVPGVGVPGAGVPGVGVPGVGVPGVGVPG |
| | | VGVPGAGVPGVGVPGVGVPGVGVPGVGVPGAGVPGVGVPGVGVPGVGVPGVGVPGAG |
| | | VPGVGVPGVGVPGVGVPGVGVPGAGVPGVGVPGVGVPGVGVPGVGVPGAGVPGVGVP |
| | | GVGVPTCSQDEFRCHDGKCISRQFVCDSDRDCLDGSDEASCPGY |
| Fusion | 17 | SKGPGVGPVGAGPVGVGPVGAGPVGVGPVGVGPVGAGPVGVGPVGAGPVGVGPVGVG |
| Protein | | PVGAGPVGVGPVGAGPVGVGPVGVGPVGAGPVGVGPVGAGPVGVGPVGVGPVGAGPV |
| | | GVGPVGAGPVGVGPVGVGPVGAGPVGVGPVGAGPVGVGPVGVGPVGAGPVGVGPVGA |
| | | GPVGVGPVGVGPVGAGPVGVGPVGAGPVGVGPVGVGPVGAGPVGVGPVGAGPVGVGP |
| | | VGVGPVGAGPVGVGPVGAGPVGVGPVGVGPVGAGPVGVGPVGAGPVGVGPVGVGPVG |
| | | AGPVGVGPVGAGPVGVGPVGVGPVGAGPVGVGPVGAGPVGVGPVGVGPVGAGPVGVG |
| | | PVGAGPVGVGPVTCSQDEFRCHDGKCISRQFVCDSDRDCLDGSDEASCPGY |
| Fusion | 18 | MSKGPGVGVPGLGVPGVGVPGLGVPGVGVPGVGVPGLGVPGVGVPGLGVPGVGVPGV |
| Protein | | GVPGLGVPGVGVPGLGVPGVGVPGVGVPGLGVPGVGVPGLGVPGVGVPGVGVPGLGVP |
| | | GVGVPGLGVPGVGVPGVGVPGLGVPGVGVPGLGVPGVGVPGVGVPGLGVPGVGVPGLG |
| | | VPGVGVPGVGVPGLGVPGVGVPGLGVPGVGVPGVGVPGLGVPGVGVPGLGVPGVGVPG |
| | | VGVPGLGVPGVGVPGLGVPGVGVPGVGVPGLGVPGVGVPGLGVPGVGVPGVGVPGLGV |
| | | PGVGVPGLGVPGVGVPGVGVPGLGVPGVGVPGLGVPGVGVPGVGVPGLGVPGVGVPGL |
| | | GVPGVGVPGVGVPGLGVPGVGVPGLGVPGVGVPGVGVPGLGVPGVGVPGLGVPGVGVP |
| | | TCSQDEFRCHDGKCISRQFVCDSDRDCLDGSDEASCPGY |
| Fusion | 19 | MSKGPGVGVPGVGVPGAGVPGVGVPGVGVPGVGVPGVGVPGAGVPGVGVPGVGVPGV |
| Protein | | GVPGVGVPGAGVPGVGVPGVGVPGVGVPGVGVPGAGVPGVGVPGVGVPGVGVPGVGV |
| | | PGAGVPGVGVPGVGVPGVGVPGVGVPGAGVPGVGVPGVGVPGVGVPGVGVPGAGVPG |
| | | VGVPGVGVPGVGVPGVGVPGAGVPGVGVPGVGVPGVGVPGVGVPGAGVPGVGVPGVG |
| | | VPGVGVPGVGVPGAGVPGVGVPGVGVPGVGVPGVGVPGAGVPGVGVPGVGVPGVGVP |
| | | GVGVPGAGVPGVGVPGVGVPGVGVPGVGVPGAGVPGVGVPGVGVPGVGVPGVGVPGA |
| | | GVPGVGVPGVGVPGVGVPGVGVPGAGVPGVGVPGVGVPGVGVPGVGVPGAGVPGVGV |
| | | PGVGVPTCSQDEFRCHDGKCISRQFVCDSDRDCLDGSDEASCPGY |
| Fusion | 20 | MSKGPGVGPVGAGPVGVGPVGAGPVGVGPVGVGPVGAGPVGVGPVGAGPVGVGPVGV |
| Protein | | GPVGAGPVGVGPVGAGPVGVGPVGVGPVGAGPVGVGPVGAGPVGVGPVGVGPVGAGP |
| | | VGVGPVGAGPVGVGPVGVGPVGAGPVGVGPVGAGPVGVGPVGVGPVGAGPVGVGPVG |
| | | AGPVGVGPVGVGPVGAGPVGVGPVGAGPVGVGPVGVGPVGAGPVGVGPVGAGPVGVG |
| | | PVGVGPVGAGPVGVGPVGAGPVGVGPVGVGPVGAGPVGVGPVGAGPVGVGPVGVGPV |
| | | GAGPVGVGPVGAGPVGVGPVGVGPVGAGPVGVGPVGAGPVGVGPVGVGPVGAGPVGV |
| | | GPVGAGPVGVGPVTCSQDEFRCHDGKCISRQFVCDSDRDCLDGSDEASCPGY |
| Linker | 21 | (EAAAK)ₙ, n = 1-20 |
| Linker | 22 | (G4S)ₙ, n = 1-30 |
| Linker | 23 | (SGGG)ₙ, n = 1-50 |
| Linker | 24 | (GGGS)ₙ, n = 1-20 |
| Linker | 25 | KESGSVSSEQLAQFRSLD |
| Linker | 26 | EGKSSGSGSESKST |
| Linker | 27 | (G)ₙ, n = 1-30 |
| Linker | 28 | GKSSGSGSESKS |
| Linker | 29 | GSTSGSGKSSEGKG |
| Linker | 30 | GSTSGSGKSSEGSGSTKG |
| Linker | 31 | GSTSGSGKPGSGEGSTKG |
| Linker | 32 | EGKSSGSGSESKEF |
| Linker | 33 | SRSSG |
| Linker | 34 | SGSSC |
| Linker | 35 | EGRGSLLTCGDVEENPGP |
| Linker | 36 | ATNFSLLKQAGDVEENPGP |
| Linker | 37 | QCTNYALLKLAGDVESNPGP |
| Linker | 38 | VKQTLNFDLLKLAGDVESNPGP |
| Polypeptide | 39 | GVGVPGVGVPGAGVPGVGVPGVGVPGVGVPGVGVPGAGVPGVGVPGVGVPGVGVPGV |
| with Phase | | GVPGAGVPGVGVPGVGVPGVGVPGVGVPGAGVPGVGVPGVGVPGVGVPGVGVPGAGV |
| Behavior | | PGVGVPGVGVPGVGVPGVGVPGAGVPGVGVPGVGVPGVGVPGVGVPGAGVPGVGVPG |
| (Reagent 2 of | | VGVPGVGVPGVGVPGAGVPGVGVPGVGVPY |
| Example 3 - | | |
| Amino Acid | | |
| Sequence; | | |
| 100V40) | | |
| (Nucleotide | 40 | GGTGTAGGCGTCCCGGGAGTAGGCGTCCCGGGGGCCGGAGTGCCGGGCGTCGGGGTC |
| Sequence; | | CCCGGAGTGGGCGTGCCCGGGGTAGGGGTTCCCGGTGTAGGAGTCCCGGGCGCTGGT |
| 100V40) | | GTACCGGGGGTCGGAGTACCGGGCGTGGGTGTGCCCGGTGTTGGTGTCCCCGGTGTG |
| | | GGAGTGCCGGGCGCTGGCGTACCTGGTGTGGGAGTACCTGGGGTCGGCGTTCCGGGC |
| | | GTTGGAGTCCCCGGTGTGGGCGTTCCTGGTGCGGGCGTTCCGGGAGTCGGAGTGCCTG |
| | | GGGTGGGAGTGCCGGGGGTCGGTGTACCCGGTGTGGGTGTCCCCGGAGCGGGGGTAC |
| | | CGGGCGTTGGCGTACCCGGCGTCGGGGTTCCGGGCGTTGGCGTACCTGGAGTCGGCG |
| | | TGCCCGGCGCGGGCGTCCCGGGCGTCGGCGTACCGGGAGTAGGTGTTCCCGGGGTCG |
| | | GAGTCCCGGGCGTAGGTGTCCCGGGCGCGGGCGTCCCCGGGGTCGGAGTGCCTGGAG |
| | | TGGGCGTCCCCGGAGTGGGCGTGCCCGGAGTAGGCGTTCCGGGGGCGGGAGTACCCG |
| | | GCGTGGGAGTACCGGGCGTAGGGGTGCCA |
| Polypeptide | 41 | GVGVPGVGVPGAGVPGVGVPGVGVPGVGVPGVGVPGAGVPGVGVPGVGVPGVGVPGV |
| with Phase | | GVPGAGVPGVGVPGVGVPGVGVPGVGVPGAGVPGVGVPGVGVPGVGVPGVGVPGAGV |
| Behavior | | PGVGVPGVGVPGVGVPGVGVPGAGVPGVGVPGVGVPGVGVPGVGVPGAGVPGVGVPG |
| | | VGVPGVGVPGVGVPGAGVPGVGVPGVGVP |
| Polypeptide | 42 | GVGPVGAGPVGVGPVGAGPVGVGPVGVGPVGAGPVGVGPVGAGPVGVGPVGVGPVGA |
| with phase | | GPVGVGPVGAGPVGVGPVGVGPVGAGPVGVGPVGAGPVGVGPVGVGPVGAGPVGVGP |
| behavior | | VGAGPVGVGPVGVGPVGAGPVGVGPVGAGPVGVGPVGVGPVGAGPVGVGPVGAGPVG |
| (50A80) | | VGPVGVGPVGAGPVGVGPVGAGPVGVGPVGVGPVGAGPVGVGPVGAGPVGVGPVGVG |
| | | PVGAGPVGVGPVGAGPVGVGPVGVGPVGAGPVGVGPVGAGPVGVGPVGVGPVGAGPV |
| | | GVGPVGAGPVGVGPVGVGPVGAGPVGVGPVGAGPVGVGPVGVGPVGAGPVGVGPVGA |
| | | GPVGVGPV |

### EXAMPLES

### Example 1. The Polypeptides with Phase Behavior and/or Fusion Proteins Described Herein Block or Enhance Lentiviral Transduction

Purpose: The ability of polypeptides with phase behavior or fusion proteins comprising polypeptides with phase behavior and the CR3 domain of the low-density lipoprotein receptor (LDLR) (Reagents 1-5 of Table B) to affect lentiviral transduction was evaluated. Each fusion protein of SEQ ID NOS: 3-5 contained a different polypeptide with phase behavior.

TABLE B

| Reagent | Description |
|---|---|
| 1 (SEQ ID NO: 1) | Polypeptide with phase behavior |
| 2 (SEQ ID NO: 2) | Polypeptide with phase behavior |
| 3 (SEQ ID NO: 3) | Fusion Protein containing a polypeptide with |
| | phase behavior (SEQ ID NO: 1) and the CR3 |
| | domain of LDLR (SEQ ID NO: 14) |
| 4 (SEQ ID NO: 4) | Fusion Protein containing a polypeptide with |
| | phase behavior (SEQ ID NO: 2) and the CR3 |
| | domain of LDLR (SEQ ID NO: 14) |
| 5 (SEQ ID NO: 5) | Fusion Protein containing a polypeptide with |
| | phase behavior (SEQ ID NO: 42) and the CR3 |
| | domain of LDLR (SEQ ID NO: 14) |

Methods—Cell Culture: HEK293T cells were plated on a 24 well plate. 500 μL of cells suspended in complete media (Dulbecco's Modified Eagle's Medium (DMEM, GIBCO™ Catalogue No. A4192001) supplemented with 10% fetal bovine serum (FBS, GIBCO™ Catalogue No. 10082147), 1× non-essential amino acid (NEAA, GIBCO™ Catalogue No. 11140050)). 50,000 cells were added to each well. The plate was incubated overnight in an incubator at 37° C., 5% CO₂.

Methods—Lentivirus Transduction: The next morning the media of the cells was replaced with 500 μL of fresh media. 5 μL of lentivirus (4×10⁶ vg/mL) encoding a green fluorescent protein (GFP) was added to each well. Reagent 1, 2, 3, 4, or 5 was added to each well (1 μM). HEK293T cells transduced with lentivirus, which were not incubated with Reagent 1, 2, 3, 4, or 5, served as a control. The lentivirus was incubated with the cells for 24 hours. A p24 ELISA was evaluated to determine the viral load. Flow cytometry was performed to identify the amount of cells expressing GFP.

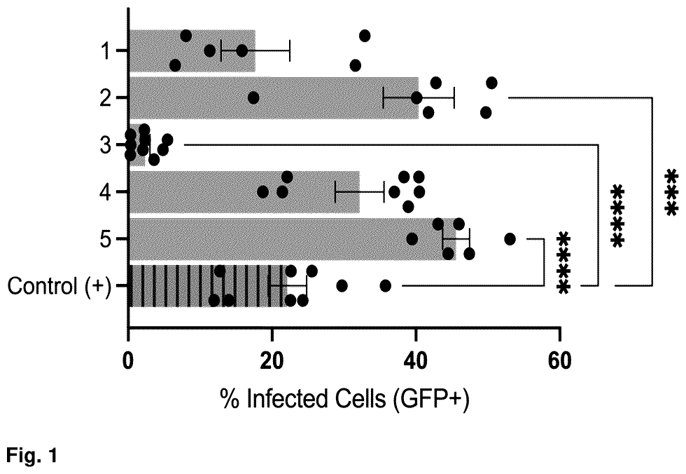

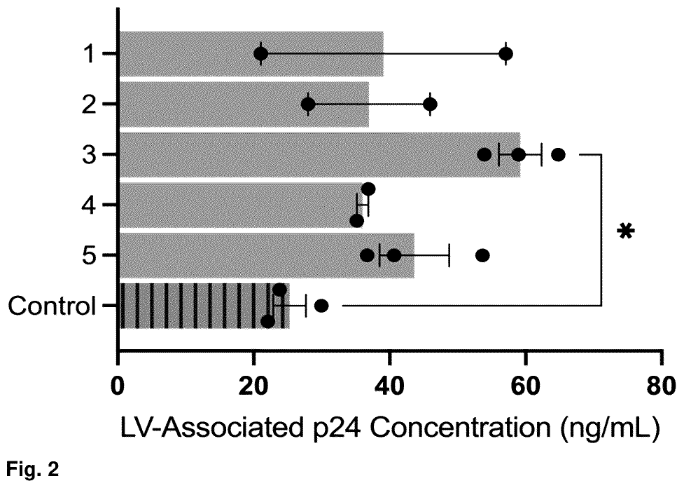

Results: FIG. 1 shows the percentage of cells expressing GFP in comparison to the control. Reagents 1 and 4 had no effect on the transduction efficiency. Reagents 2 and 5 significantly enhanced lentiviral transduction efficiency. In contrast, Reagent 3 significantly blocked lentiviral transduction efficiency. Notably, Reagents 3 and 5 contained different polypeptides with phase behavior. Reagent 3 comprises the polypeptide with phase behavior of SEQ ID NO: 1, and Reagent 5 contained the polypeptide with phase behavior of SEQ ID NO: 11. This data shows that unexpectedly different polypeptides with phase behavior have different effects on lentiviral transduction. The viral load in the supernatant of each sample remained consistent (FIG. 2). This indicates that virus was retained in the supernatant without incurring damage.

### Example 2. The Fusion Proteins Described Herein Stabilize Lentivirus in Various Conditions

Purpose: The ability of polypeptides with phase behavior from Example 1 (Reagents 4 and 5 of Table B) to stabilize lentivirus at various conditions prior to transduction was evaluated. Each protein of SEQ ID NOS: 4 and 5 contained a different polypeptide with phase behavior.

Methods—Cell Culture: HEK293T cells were plated on a 24 well plate. 500 μL of cells suspended in complete media (Dulbecco's Modified Eagle's Medium (DMEM, GIBCO™ Catalogue No. A4192001) supplemented with 10% fetal bovine serum (FBS, GIBCO™ Catalogue No. 10082147), 1× non-essential amino acid (NEAA, GIBCO™ Catalogue No. 11140050)). 50,000 cells were added to each well. The plate was incubated overnight in an incubator at 37° C., 5% CO₂.

Methods—Lentivirus Production: Lentivirus were produced using adherent cells in media containing serum or in suspension cells in media lacking serum.

Methods—Lentivirus Transduction: The next morning the media of the cells was replaced with 500 μL of fresh media. 5 μL of lentivirus (4×10⁶ vg/mL) encoding a green fluorescent protein (GFP) was incubated with Reagent 4 or 5 (10 μM). Subsequently, the composition containing lentivirus and Reagent 4 or 5 was added to each well. HEK293T cells transduced with lentivirus, which were not incubated with Reagent 4 or 5, served as a control. The lentivirus was incubated with the cells for 24 hours. Flow cytometry was performed to identify the amount of cells expressing GFP.

Methods—Stabilizing at Various Temperatures: Aliquots containing Reagent 4 or 5 mixed with lentivirus were stored at: 1) 4-8° C. for 2 months, with additional samples taken weekly for testing; 2) 37° C. for 6 hours, with additional samples taken hourly for testing; or 3) −80° C. for one hour and then thawed on ice (one freeze-thaw cycle), with freeze-thaw cycle repeated for 5 cycles in total, with additional samples taken after each freeze-thaw cycle for testing. Samples taken at various time points were transduced as previously detailed.

Methods—Stabilizing at High Salt Concentration: Aliquots containing Reagent 4 or 5 mixed with lentivirus were stored and 1.5 M NaCl were rotated at room temperature for 12 hours, with additional samples taken hourly for testing. Samples taken at various time points were transduced with lentivirus as previously detailed.

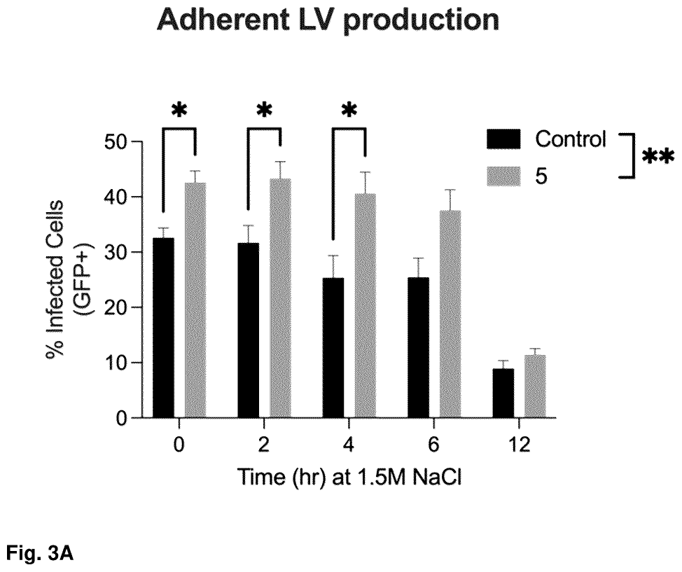

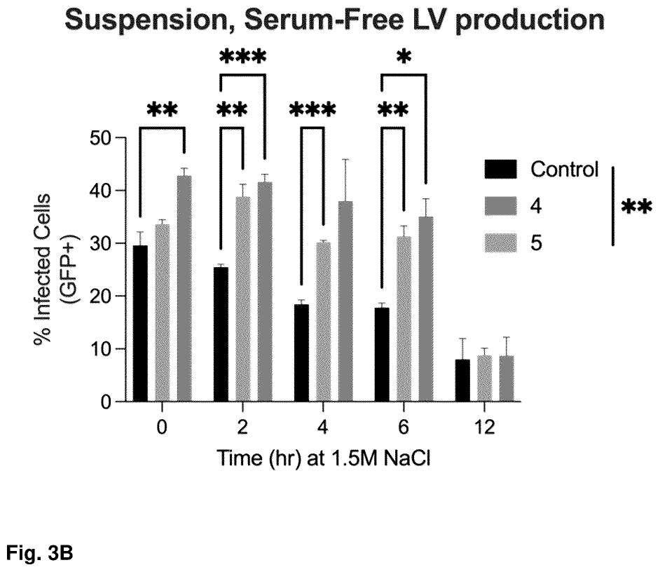

Results: FIGS. 3A and 3B show stability of lentiviral particles in 1.5 M NaCl from 0 to 12 hours as measured by percent GFP+ cells. FIG. 3A shows the stability of lentivirus produced in adherent cells in the presence of serum and Reagent 5, as compared to control. FIG. 3B shows the stability of lentivirus produced in suspension cells in the absence of serum and in the presence of Reagent 4 or 5, as compared to control. The CR3 domain of LDLR provided enhanced, statistically significant stability up to 6 hours as compared to the control for both lentivirus produced with adherent and suspension cells.

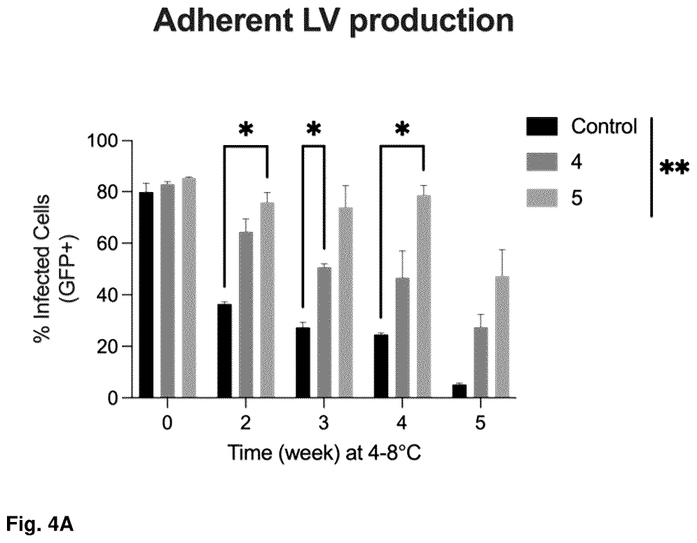

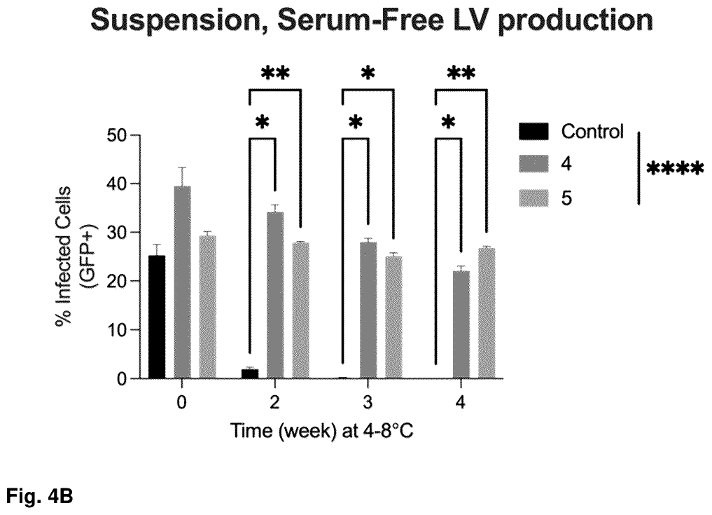

FIGS. 4A and 4B show stability of lentiviral particles at 4-8° C. for 2 months as measured by percent GFP+ cells. FIG. 4A shows the stability of lentivirus produced in adherent cells in the presence of serum and Reagent 4 or 5, as compared to control. FIG. 4B shows the stability of lentivirus produced in suspension cells, produced in the absence of serum, and in the presence of Reagent 4 or 5, as compared to control. The CR3 domain of LDLR provided enhanced stability up to 5 weeks, as compared to the control for both adherent and suspension conditions.

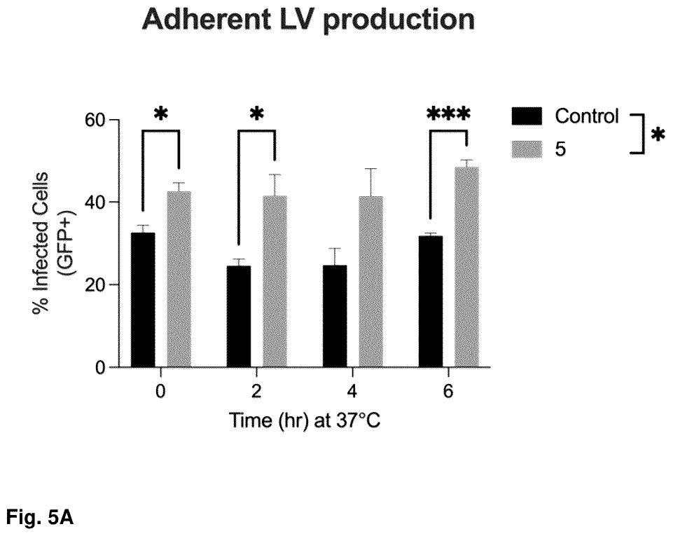

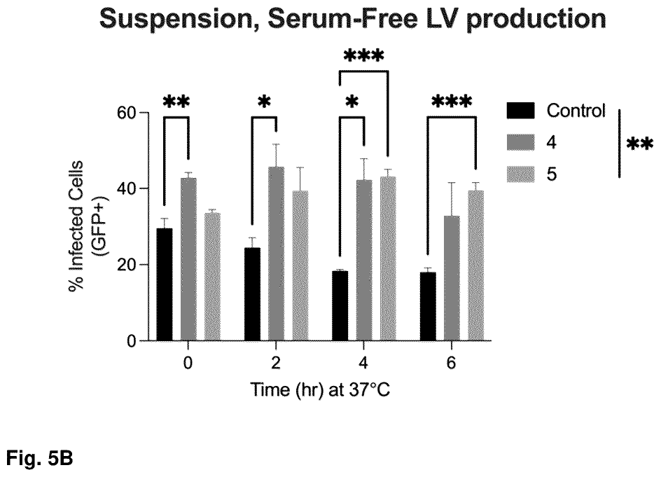

FIGS. 5A and 5B show stability of lentiviral particles at 37° C. for 6 hours as measured by percent GFP+ cells. FIG. 5A shows the stability of lentivirus produced in adherent cells in the presence of serum and Reagent 5 versus control. FIG. 5B shows the stability of lentivirus produced in suspension cells in the absence of serum and in the presence of Reagent 4 or 5, as compared to control. The CR3 domain of LDLR provided enhanced, statistically significant stability up to 6 hours as compared to the control for both adherent and suspension conditions.

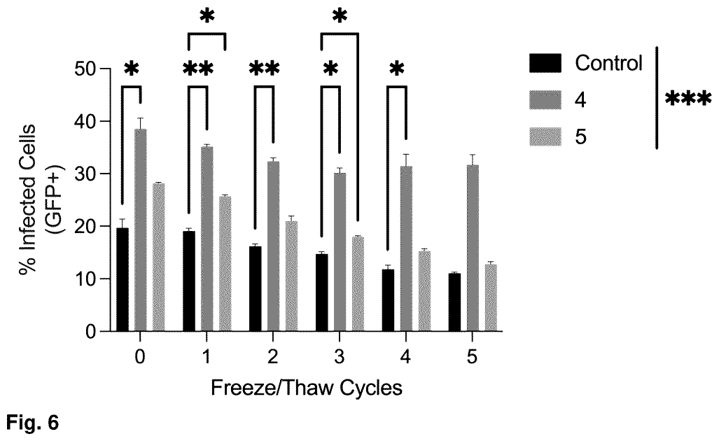

FIG. 6 shows stability of lentiviral particles at −80° C. for 5 freeze-thaw cycles as measured by percent GFP+ cells in suspension, serum-free conditions for lentiviral production with Reagent 4 or 5 versus control. The CR3 domain of LDLR provided enhanced, statistically significant stability up to 5 freeze-thaw cycles as compared to the control.

These results show that the addition of Reagent 4 and/or 5 stabilizes lentivirus exposed to various temperatures (4-8° C., 37° C., and −80° C.) and salt conditions (1.5 M NaCl), thus retaining lentivirus efficacy.

### Example 3. The Polypeptides with Phase Behavior Described Herein Block Adenoviral Transduction

Purpose: The ability of polypeptides with phase behavior (Reagents 1 and 2 of Table C) to influence adenovirus transduction was evaluated.

TABLE C

| Reagent | Description |
|---|---|
| 1 | a polypeptide with phase behavior (SEQ ID |
| | NO: 1) |
| 2 | a polypeptide with phase behavior (SEQ ID |
| | NO: 39) |

Methods—Cell Culture: HEK293T cells were plated on a 24 well plate. 500 μL of cells suspended in complete media (Dulbecco's Modified Eagle's Medium (DMEM, GIBCO™ Catalogue No. A4192001) supplemented with 10% fetal bovine serum (FBS, GIBCO™ Catalogue No. 10082147), 1× non-essential amino acid (NEAA, GIBCO™ Catalogue No. 11140050)). 100,000 cells were added to each well. The plate was incubated overnight in an incubator at 37° C., 5% CO₂.

Methods—Adenovirus Transduction: The next morning the media of the cells was replaced with 500 μL of fresh media. 1 μL of adenovirus (1×10¹² vg/mL) encoding a green fluorescent protein (GFP) was added to each well. Reagent 1 or 2 was added to each well (0.1 μM, 1 μM, 10 μM, 100 μM). HEK293T cells transduced with adenovirus, which were not incubated with Reagent 1 or 2, served as a control. The adenovirus was incubated with the cells for 24 hours. An INVITROGEN™ EVOS™ M5000 Live Cell Imager was used to verify infectivity. Flow cytometry was performed to identify the amount of cells expressing GFP.

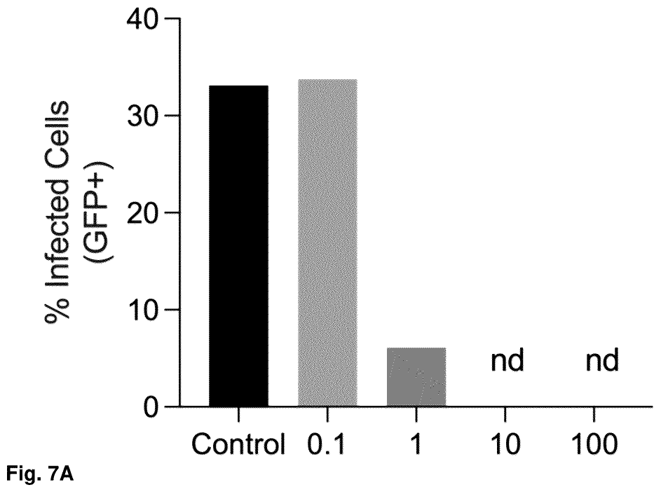

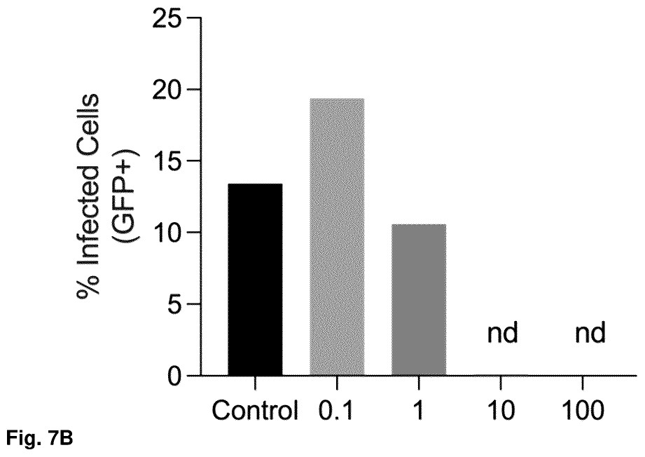

Results: FIG. 7A and FIG. 7B show the percentage of cells expressing GFP in comparison to the control for Reagent 1 and 2, respectively. Reagents 1 and 2 both blocked adenoviral transduction at concentrations greater than 1 μM (not determined at higher concentrations).

### NUMBERED EMBODIMENTS OF THE DISCLOSURE

Notwithstanding the appended claims, the disclosure sets forth the following numbered embodiments:

1. A fusion protein comprising the CR3 domain of the low density lipoprotein (LDLR) that is at least 80% identical to the polypeptide of SEQ ID NO: 14 and a polypeptide with phase behavior that is at least 80% identical to a polypeptide of any one of SEQ ID NOS: 1, 2, or 11-13.

2. The fusion protein of embodiment 1, wherein the CR3 domain of the LDLR comprises a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 14.

3. The fusion protein of embodiment 2, wherein the CR3 domain of the LDLR comprises the polypeptide sequence of SEQ ID NO: 14.

4. The fusion protein of any one of embodiments 1-3, wherein the polypeptide with phase behavior comprises a polypeptide that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 2, or 11-13.

4. The fusion protein of any one of embodiments 1-3, wherein the polypeptide with phase behavior comprises the polypeptide sequence of SEQ ID NO: 1.

5. The fusion protein of any one of embodiments 1-3, wherein the polypeptide with phase behavior comprises the polypeptide sequence of SEQ ID NO: 2.

6. The fusion protein of any one of embodiments 1-3, wherein the polypeptide with phase behavior comprises the polypeptide sequence of SEQ ID NO: 11.

7. The fusion protein of any one of embodiments 1-6, wherein the polypeptide with phase behavior is N-terminal to the CR3 domain of the LDLR.

8. The fusion protein of any one of embodiments 1-6, wherein the polypeptide with phase behavior is C-terminal to the CR3 domain of the LDLR.

9. The fusion protein of any one of embodiments 1-6, comprising a linker between the polypeptide with phase behavior and the CR3 domain of the LDLR.

10. The fusion protein of embodiment 9, wherein the linker has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 21-38.

11. A method of improving lentiviral transduction efficiency, comprising transducing a cell with a lentivirus and administering a fusion protein of any one of embodiments 1-10.

12. The method of embodiment 11, wherein the lentiviral transduction efficiency is improved compared to the lentiviral transduction efficiency of a method, which does not comprise administering a fusion protein of any one of embodiments 1-10.

13. The method of embodiment 11, wherein the lentiviral transduction efficiency is improved compared to a conventional method for improving lentiviral transduction.

14. The method of any one of embodiments 11-13, wherein lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%.

15. The method of any one of embodiments 11-14, wherein the fusion protein comprises a polypeptide with phase behavior with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 11.

16. The method of embodiment 15, wherein the fusion protein comprises a polypeptide with phase behavior having the amino acid sequence of SEQ ID NO: 11.

17. The method of any one of embodiments 11-16, wherein the fusion protein has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 5.

18. The method of embodiment 17, wherein the fusion protein has the amino acid sequence of SEQ ID NO: 5.

19. The method of any one of embodiments 14-18, wherein the lentiviral transduction efficiency is improved by at least 120%.

20. A method of improving lentiviral transduction efficiency, comprising transducing a cell with a lentivirus and administering a polypeptide with phase behavior; wherein the amino acid sequence of the polypeptide with phase behavior is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2.

21. The method of embodiment 20, wherein the polypeptide with phase behavior has the amino acid sequence of SEQ ID NO: 2.

22. The method of embodiment 20 or 21, wherein viral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%.

23. The method of any one of embodiments 20-22, wherein the lentiviral transduction efficiency is improved by at least 90%.

24. The method of any one of embodiments 20-23, wherein the lentiviral transduction efficiency is improved compared to the viral transduction efficiency of a method, which does not comprise administering the polypeptide with phase behavior.

25. The method of any one of embodiments 20-23, wherein the lentiviral transduction efficiency is improved compared to a conventional method for improving lentiviral transduction.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that it constitutes valid prior art or form part of the common general knowledge in any country in the world. The following references are incorporated by reference herein in their entireties: U.S. Pat. No. 11,015,174 and U.S. Publication No. 2019/0055523.

What is claimed is:

1. A fusion protein comprising the CR3 domain of the low density lipoprotein (LDLR) that is at least 80% identical to the polypeptide of SEQ ID NO: 14 and a polypeptide with phase behavior that is at least 80% identical to a polypeptide of any one of SEQ ID NOS: 1, 2, 11-13, and 41-42.

2. The fusion protein of claim 1, wherein the CR3 domain of the LDLR comprises a polypeptide that is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the polypeptide of SEQ ID NO: 14.

3. The fusion protein of claim 1, wherein the CR3 domain of the LDLR comprises the polypeptide sequence of SEQ ID NO: 14.

4. The fusion protein of claim 1, wherein the polypeptide with phase behavior comprises a polypeptide that is at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 1, 2, 11-13, and 41-42.

5. The fusion protein of claim 1, wherein the polypeptide with phase behavior comprises the polypeptide sequence of any one of SEQ ID NO: 1, SEQ ID NO: 12, SEQ ID NO: 13, and SEQ ID NO: 42.

6. The fusion protein of claim 1, comprising a linker between the polypeptide with phase behavior and the CR3 domain of the LDLR.

7. The fusion protein of claim 6, wherein the linker has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of any one of SEQ ID NOS: 21-38.

8. The fusion protein of claim 1, wherein the fusion protein has an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to a polypeptide of SEQ ID NO: 5.

9. A method of improving lentiviral transduction efficiency, comprising transducing a cell with a lentivirus and administering a fusion protein of claim 1, wherein lentiviral transduction of the cells administered the lentivirus and the fusion protein is improved as compared to cells that are transduced with the lentivirus and not administered the fusion protein.

10. The method of claim 9, wherein lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%.

11. The method of claim 9, wherein the fusion protein comprises a polypeptide with phase behavior with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 12 or SEQ ID NO: 42.

12. The method of claim 11, wherein the fusion protein comprises a polypeptide with phase behavior having the amino acid sequence of SEQ ID NO: 12 or SEQ ID NO: 42.

13. The method of claim 9, wherein the CR3 domain of the LDLR is a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 14.

14. The method of claim 9, wherein the wherein the CR3 domain of the LDLR is a polypeptide of SEQ ID NO: 14.

15. The method of claim 9, wherein the fusion protein has an amino acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 5.

16. The method of claim 9, wherein the lentiviral transduction efficiency is improved by at least 120%.

17. A method of improving lentiviral transduction efficiency, comprising transducing a cell with a lentivirus and administering a polypeptide with phase behavior; wherein the amino acid sequence of the polypeptide with phase behavior is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 12, wherein lentiviral transduction of the cells administered the lentivirus and the polypeptide with phase behavior is improved as compared to cells that are administered the lentivirus and not administered the polypeptide with phase behavior.

18. The method of claim 17, wherein lentiviral transduction efficiency is improved by at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, or at least 500%.

19. The method of claim 17, wherein the lentiviral transduction efficiency is improved by at least 90%.

20. The method of claim 17, wherein the polypeptide with phase behavior has the amino acid sequence of SEQ ID NO: 12.


SEQUENCE LISTING (42 sequences)

SEQ ID NO: 1 - protein, 401 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPY

SEQ ID NO: 2 - protein, 401 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPY

SEQ ID NO: 3 - protein, 439 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 4 - protein, 439 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 5 - protein, 389 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 6 - DNA, 831 bp, synthetic construct
  source 1..831: mol_type = other DNA
tagccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacancgacgcccggaacacccagccccggaacgcccacacca

SEQ ID NO: 7 - DNA, 804 bp, synthetic construct
  source 1..804: mol_type = other DNA
tagcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccangaacaccaacgcccggtacgcccacacctggg

SEQ ID NO: 8 - DNA, 720 bp, synthetic construct
  source 1..720: mol_type = other DNA
tagcccgggcagctggcttcatcgctgccatccagacaatcgcgatcgctatcgcaaacgaactggcggctgatgcatttgccatcatgacagcggaactcatcttggctgcacgtgccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccagggacgccaacacccggtacacccagacctggcacaccgacgcccggaacacccagccccggaacgcccacaccanggacgccaacacccggtacacccagacctggcacaccgacgcccggaacacccagccccggaacgcccacaccnngnacgccaacacccggtacacccagacctggcacaccgacgcccggaacacccagccccggaacgcccacaccn

SEQ ID NO: 9 - DNA, 809 bp, synthetic construct
  source 1..809: mol_type = other DNA
tagcccgggcagctggcttcatcgctgccatccagacaatcgcgatcgctatcgcaaacgaactggcggctgatgcatttgccatcatgacagcggaactcatcttggctgcacgtgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccaggaacaccaacgcccggtacgcccacacctgggacacctacgcccggaacacccacgcccggcacgccgacaccangaacaccaacgcccggtacgcccacacctgggacacctacgcccggaacacccacgcccggcacgccgacaccangaacaccaacgcccggtacgcccacacctgggacacctacgcccggaacacccacgcccggcacgccgacacc

SEQ ID NO: 10 - DNA, 765 bp, synthetic construct
  source 1..765: mol_type = other DNA
tagcccgggcagctggcttcatcgctgccatccagacaatcgcgatcgctatcgcaaacgaactggcggctgatgcatttgccatcatgacagcggaactcatcttggctgcacgtgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggaacgcctgctcctggaacacccacgcctggcactccggcgccaggaacacccacacctggnacgcctgctcctggaacacccacgcct

SEQ ID NO: 11 - protein, 400 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPV

SEQ ID NO: 12 - protein, 400 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVP

SEQ ID NO: 13 - protein, 400 aa, synthetic construct
GVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVP

SEQ ID NO: 14 - protein, 39 aa, synthetic construct
TCSQDEFRCHDGKCISRQFVCDSDRDCLDGSDEASCPGY

SEQ ID NO: 15 - protein, 443 aa, synthetic construct
SKGPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 16 - protein, 443 aa, synthetic construct
SKGPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 17 - protein, 393 aa, synthetic construct
SKGPGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 18 - protein, 444 aa, synthetic construct
MSKGPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPGVGVPGLGVPGVGVPGLGVPGVGVPTCSQDEFRCHDGKCISRQFVCDSDRDCLDGSDEASCPGY

SEQ ID NO: 19 - protein, 444 aa, synthetic construct
MSKGPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPTCSQDEFRCHDGKCISRQFVCDSDRDCLDGSDEASCPGY

SEQ ID NO: 20 - protein, 394 aa, synthetic construct
MSKGPGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVTCSQDEFRCHDGKCISRQFVCDSDRDCLDGSDEASCPGY

SEQ ID NO: 21 - protein, 100 aa, synthetic construct
  REGION 1..100: note = Linker peptide
  REPEAT 1..5: note = single repeat of (EAAAK)n where n=1-20
  VARIANT 6..100: note = up to 19 copies of (EAAAK) can be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 22 - protein, 150 aa, synthetic construct
  REGION 1..150: note = Linker peptide
  REPEAT 1..5: note = single repeat of (GGGGS)n where n=1-30
  VARIANT 6..150: note = up to 29 copies of (GGGGS) can be deleted
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 23 - protein, 200 aa, synthetic construct
  REGION 1..200: note = Linker peptide
  REPEAT 1..4: note = single repeat of (SGGG)n where n=1-50
  VARIANT 5..200: note = up to 49 copies of (SGGG) can be deleted
SGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGG

SEQ ID NO: 24 - protein, 80 aa, synthetic construct
  REGION 1..80: note = Linker peptide
  REPEAT 1..4: note = single repeat of (GGGS)n where n=1-20
  VARIANT 5..80: note = up to 19 copies of (SGGG) can be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 25 - protein, 18 aa, synthetic construct
KESGSVSSEQLAQFRSLD

SEQ ID NO: 26 - protein, 14 aa, synthetic construct
EGKSSGSGSESKST

SEQ ID NO: 27 - protein, 30 aa, synthetic construct
  REGION 1..30: note = Linker peptide
  REPEAT 1: note = single repeat of (G)n where n=1-30
  VARIANT 2..30: note = up to 29 copies of (G) can be deleted
GGGGGGGGGGGGGGGGGGGGGGGGGGGGGG

SEQ ID NO: 28 - protein, 12 aa, synthetic construct
GKSSGSGSESKS

SEQ ID NO: 29 - protein, 14 aa, synthetic construct
GSTSGSGKSSEGKG

SEQ ID NO: 30 - protein, 18 aa, synthetic construct
GSTSGSGKSSEGSGSTKG

SEQ ID NO: 31 - protein, 18 aa, synthetic construct
GSTSGSGKPGSGEGSTKG

SEQ ID NO: 32 - protein, 14 aa, synthetic construct
EGKSSGSGSESKEF

SEQ ID NO: 33 - protein, 5 aa, synthetic construct
SRSSG

SEQ ID NO: 34 - protein, 5 aa, synthetic construct
SGSSC

SEQ ID NO: 35 - protein, 18 aa, synthetic construct
EGRGSLLTCGDVEENPGP

SEQ ID NO: 36 - protein, 19 aa, synthetic construct
ATNFSLLKQAGDVEENPGP

SEQ ID NO: 37 - protein, 20 aa, synthetic construct
QCTNYALLKLAGDVESNPGP

SEQ ID NO: 38 - protein, 22 aa, synthetic construct
VKQTLNFDLLKLAGDVESNPGP

SEQ ID NO: 39 - protein, 201 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPY

SEQ ID NO: 40 - DNA, 600 bp, synthetic construct
  source 1..600: mol_type = other DNA
ggtgtaggcgtcccgggagtaggcgtcccgggggccggagtgccgggcgtcggggtccccggagtgggcgtgcccggggtaggggttcccggtgtaggagtcccgggcgctggtgtaccgggggtcggagtaccgggcgtgggtgtgcccggtgttggtgtccccggtgtgggagtgccgggcgctggcgtacctggtgtgggagtacctggggtcggcgttccgggcgttggagtccccggtgtgggcgttcctggtgcgggcgttccgggagtcggagtgcctggggtgggagtgccgggggtcggtgtacccggtgtgggtgtccccggagcgggggtaccgggcgttggcgtacccggcgtcggggttccgggcgttggcgtacctggagtcggcgtgcccggcgcgggcgtcccgggcgtcggcgtaccgggagtaggtgttcccggggtcggagtcccgggcgtaggtgtcccgggcgcgggcgtccccggggtcggagtgcctggagtgggcgtccccggagtgggcgtgcccggagtaggcgttccgggggcgggagtacccggcgtgggagtaccgggcgtaggggtgcca

SEQ ID NO: 41 - protein, 200 aa, synthetic construct
GVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVPGVGVPGVGVPGAGVPGVGVPGVGVP

SEQ ID NO: 42 - protein, 350 aa, synthetic construct
GVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPVGVGPVGAGPVGVGPVGAGPVGVGPV